(19) United States
(12) Patent Application Publication
(10) Pub. No.: US 2014/0341922 A1
(43) Pub. Date: Nov. 20, 2014

(54) Method for Overcoming Tolerance to Targeted Anti-Cancer Agent

(71) Applicant: SUN R&DB Foundation, Seoul (KR)

(72) Inventor: Ho Young Lee, Gwanak-gu (KR)

(73) Assignee: SUN R & D Foundation

(21) Appl. No.: 14/360,117

(22) PCT Filed: Nov. 23, 2012
(86) PCT No.: PCT/KR2012/009991

§ 371 Date: May 22, 2014

(30) Foreign Application Priority Data
Nov. 25, 2011 (KR) 10-2011-0124207
Nov. 23, 2012 (KR) 10-2011-0133739

(51) Int. Cl. C07K 16/28; A61K 31/519; G01N 33/574; A61K 39/395

(52) U.S. Cl. CPC C07K 16/2848; C07K 16/2863; A61K 39/39558; A61K 39/3955; A61K 31/519; G01N 33/57492
USPC 424/142.1; 530/388.22; 435/7.23

(57) ABSTRACT

Provided are a pharmaceutical composition for suppressing a resistance to a targeted anticancer drug, which at least one selected from the group consisting of an integrin β3 neutralizing antibody, integrin β3 siRNA, Src inhibitor, and Src siRNA as an active ingredient, and an anticancer supplement. The pharmaceutical composition may increase an anticancer therapeutic effect when administered in combination with a conventional targeted anticancer drug. In addition, the pharmaceutical composition is expected to be used in development of an integrin β3-targeted targeted anticancer drug.

### TECHNICAL FIELD

The present invention relates to a method of overcoming the resistance to a targeted anticancer drug, and particularly, to a method of overcoming the resistance to an insulin-like growth factor-1 receptor (IGF-1R) targeted anticancer drug.

### BACKGROUND ART

The IGF-1R has been reported to play an important role in tumorigenesis by promoting cell proliferation, survival, malignant transformation, angiogenesis, and invasion (Basega, 1999; Pollak et al., 2004). The IGF axis consists of two receptors (IGF-1R and IGF-2R), ligands (IGF-1, IGF-2, and insulin), and at least six IGF-binding proteins (IGFBPs) that modulate the bioavailability of IGF. Deregulation of IGF-1R-mediated signaling including production of high-level IGF, overexpression of IGF-1R, and/or expression of IGFBPs, a decrease in IGF-2R heterozygosity, and biallelic expression of IGF-2 is closely related to the increased risk of various types of cancers (Chang et al., 2002a; Chang et al., 2002b; Jamieson et al., 2003; Kim et al., 2009; Larsson et al., 2005; Moorehead et al., 2003; Papadimitrakopoulou et al., 2006; Wu et al., 2004; Zhan et al., 1995). Accordingly, the IGF receptor-mediated signal transduction system has been considered as an attractive target for developing anticancer drugs.

Several clinical trials to investigate the therapeutic efficacy of IGF-1R targeted therapies using monoclonal antibodies (mAbs) or small molecule tyrosine kinase inhibitors (TKIs) against IGF-1R have been conducted (Bahr and Groner, 2004; Garcia-Echeverria, 2006). Anti-IGF-1R mAbs were well tolerated as single agents and showed moderate side effects in phase I clinical trials (Olmos et al., 2009; Tolcher et al., 2009). Ewing's sarcoma patients treated with anti-IGF-1R mAbs including AMG-479, R1507, or figitumumab showed sporadic antitumor responses (Kurzrock et al., 2010; Olmos et al., 2010; Pappo et al., 2010; Quek et al., 2010; Tap et al., 2010). However, in recent phase II and III clinical trials in patients with recurrent or metastatic head and neck squamous cell carcinoma (HNSCC), non-small cell lung cancer (NSCLC), and colorectal cancer, anti-IGF-1R mAbs showed marginal and limited antitumor effects (Businesswire., 2009; Patel S, 2009; Reidy et al, 2010; Schmitz, 2010); however, the mechanism underlying primary and/or acquired resistance to anti-IGF-1R mAbs are poorly understood.

Integrins, a family of adhesive receptors, are composed of 8 kinds of 13 subunits and 18 kinds of a subunits (Bikle, 2008; Hynes, 2002). Activation of integrins by ligand binding mediates autophosphorylation of focal adhesion kinase (FAK) at tyrosine 397 residue (Y397). This autophosphorylation is required for p85 binding and PI3K activation (Chen et al., 1996), the recruitment of Src, and Src-dependent phosphorylation of FAK at Try861 and Tyr925 and phosphorylation of an epidermal growth factor receptor (EGFR) at Tyr845 (Alghisi and Ruegg, 2006; Desgrosellier and Cheresh, 2009; Home et al., 2005; Hynes, 2002). While N-terminal domain of FAK interacts with β1 and β3 integrins (Schaller et al., 1992; Schaller et al., 1995), C-terminal domain of FAK binds to Src homology2 and Src homology3 (SH2 and SH3) domains of several proteins (Malik and Parsons, 1996). Previous studies showed that monoclonal antibodies (mAbs) and small molecule inhibitors against αvβ3 inhibited tumor growth and angiogenesis in several animal models (Dayam et al., 2006; Kumar et al., 2001; Trikha et al., 2002). In addition, there are several reports to describe that integrin αvβ3 is involved in cell proliferation, metastasis, and drug resistance and thus stimulates tumor growth and progression in several types of cancer (Brozovic et al., 2008; Hood and Cheresh, 2002; Stefanidakis and Koivunen, 2006). In particular, a recent study demonstrates that IGF-1 directly binds to αvβ3 (Saegusa et al., 2009), suggesting a direct regulatory link between the IGF system and a specific integrin signal.

### DISCLOSURE

### Technical Problem

Although targeted anticancer drugs with improved selectivity to cancer cells have been extensively investigated in a variety of clinical trials, primary and/or acquired resistance to these targeted anticancer therapeutic agents including IGF-1R targeted therapies have been reported. Therefore, exploring alternative therapeutic approaches as well as understanding drug resistance mechanisms would be essential to develop effective anticancer treatment.

To accomplish this, the present inventor investigated a predictive biomarker to select suitable responders to IGF-1R targeted therapies using anti-IGF-1R mAbs, identified the mechanisms underlying resistance to anti-IGF-1R mAbs-based therapies, thereby providing co-targeting strategies to overcome anticancer drug resistance and completing the present invention.

The present invention is directed to provide a pharmaceutical composition and a method for overcoming resistance to targeted anticancer drugs, in particular IGF-1R-based targeted therapies.

However, the technical solutions to be accomplished are not limited to the above-described objects, and it should be fully understood from the following descriptions to those of ordinary skill in the art.

### Technical Solution

One aspect of the present invention provides a pharmaceutical composition to overcome resistance to targeted anticancer drug comprising at least one selected from the group consisting of an integrin β3 neutralizing antibody, integrin β3 siRNA, Src inhibitor, and Src siRNA as an active ingredient.

Another aspect of the present invention provides an anticancer supplement, which includes at least one selected from the group consisting of an integrin β3 neutralizing antibody, integrin β3 siRNA, Src inhibitor, and Src siRNA as an active ingredient, and which leading to increase therapeutic effects of anticancer therapies.

Still, another aspect of the present invention provides a method to improve efficacy of anticancer drugs, which comprises administration of targeted anticancer drugs in combination with at least one selected from the group consisting of an integrin β3 neutralizing antibody, integrin β3 siRNA, Src inhibitor and Src siRNA, or a pharmaceutically available salt thereof

In the present invention, the anticancer drug may be an insulin-like growth factor-1 receptor(IGF-1R) targeted anticancer drug, and preferably, an anti-IGF-1R monoclonal antibody.

In the present invention, the integrin β3 neutralizing antibody, integrin β3 siRNA, Src inhibitor, or Src siRNA serve to inhibit resistance to the targeted anticancer drug through pathways, which is inhibit insulin-like growth factor dependency of the anti-IGF-1R monoclonal antibody, inhibit phosphorylation of Src, EGFR, Akt, FAK, or mTOR, or inducing dephosphorylation of p-Src, p-EGFR, p-Aid, p-FAK, or p-TOR.

In particular, in the present invention, phosphorylation of Src may occur at tyrosine 416 (Y416), and phosphorylation of EGFR may occur at tyrosine 1068 (Y1068 and/or tyrosine 845 (Y845). In addition, phosphorylation of FAK may occur at tyrosine 861 (Y861).

### Advantageous Effects

The present invention provides a method to overcome resistance to targeted anticancer therapies including IGF-1R-targeted anticancer drugs by interrupting signal transduction pathways involved in resistance to anti-IGF-1R mAbs and similar anticancer therapies thereto. In addition, a pharmaceutical composition of the present invention can increase the anticancer therapeutic effects of other targeted anticancer drugs when administrated in combination. Moreover, the present invention is expected to be applied to integrin β3-targeted anticancer therapies in development or currently being investigated in preclinical and clinical trials.

### DESCRIPTION OF DRAWINGS

FIG. 1 shows the concentration-dependent inhibitory effect of cixutumumab on IGF-1-induced IGF-1R phosphorylation.

FIG. 2 shows the effect of cixutumumab on growth of a panel of human head and neck squamous cell carcinoma (HNSCC) and non-small cell lung cancer (NSCLC) cells grown in a conventional two-dimensional (2D) system.

FIG. 3 shows the effect of cixutumumab (25 μg/ml) on growth of HNSCC cells, such as LN686 and UMSCC38, and NSCLC cells, such as A549m, grown in media supplemented with reduced serum containing 1% FBS.

FIG. 4 shows the concentration-dependent inhibitory effects of cixutumumab on colony formation of OSC19 and A549 cells grown in a three-dimensional (3D) system using soft agar.

FIG. 5 shows minimal effect of cixutumumab on growth of colonies of cixutumumab-resistant LN686 and FADU cells in 3D-mimic 2D culture conditions using polyHEMA-coated plates (PCPs) compared to those of cixutumumab-sensitive OSC19 cells.

FIG. 6 shows changes in cell numbers of control- or cixutumumab-treated LN686, FADU, and OSC19 cells grown in 3D-mimic 2D culture conditions using a polyHEMA-coated plate (PCP) or an ultra-low attached plate (UAP) for 1, 3, 5, and 7 days.

FIG. 7 shows the effect of cixutumumab on growth of 13 HNSCC and 6 NSCLC cells grown in 3D-mimic 2D culture conditions using a polyHEMA-coated plate (PCP) and an ultra-low attached plate (UAP).

FIG. 8 shows the effect of is cixutumumab on the colony formation of 10 HNSCC and 6 NSCLC cells grown in a three-dimensional (3D) system using soft agar.

FIG. 9 shows the antitumor effect of cixutumumab in tumor xenografts models prepared by implanting HNSCC cells, such as LN686 and UMSCC38, and NSCLC cells, such as H226B and A549m, into mice.

FIG. 10 shows increase of Akt phosphorylation by treatment with cixutumumab in cixutumumab-resistant LN686 cells compared to cixutumumab-sensitive OSC19 cells in spite of complete inhibition of IGF-1-induced IGF-1R phosphorylation in both cell lines.

FIG. 11 shows difference between expression and activation of IGF-1R downstream signaling by cixutumumab in cixutumumab-resistant cells compared to cixutumumab-sensitive cells.

FIG. 12 shows increased phosphorylation of Src and EGFR at Src-specific phosphorylation (Y845) sites, by cixutumumab treatment in cixutumumab-resistant cells compared to cixutumumab-sensitive cells.

FIG. 13 shows quantification of data in FIG. 12.

FIG. 14 shows 10% FBS-mediated IGF-1R phosphorylation inhibitory activity and an effect of stimulating activation of time-dependent EGFR and its downstream signal transduction when cixutumumab is treated to LN686 cells for 0.5, 1, 3, 6, and 72 hours.

FIG. 15 shows time-dependent increased recruitment of Src and FAK to integrin β3 by cixutumumab treatment.

FIG. 16 shows a change in IGF-1R and EGFR-mediated signal transductions when the IGF-1 or cixutumumab-treating time is increased, leading to an increase in Src, FAK, Akt, and mTOR phosphorylation due to cixutumumab.

FIG. 17 shows dose-dependent increased recruitment of Src and FAK to integrin β3 by IGF-1 treatment.

FIG. 18 shows lack of increased phosphorylation of Src, Akt, and EGFR (Y845) by treatment with cixutumumab without serum stimulation in LN686 cells.

FIG. 19 shows no increased association among Src and FAK and integrin β1 by cixutumumab treatment with IGF-1 stimulation.

FIG. 20 shows time-dependent increased recruitment of Src and FAK to integrin β3 by cixutumumab treatment in the presence of serum (10% FBS) stimulation.

FIG. 21 shows increased recruitment of Src and FAK to integrin β3 but not to integrin 131 by cixutumumab treatment in the presence of IGF-1 stimulation.

FIG. 22 shows time-dependent re-phosphorylation of EGFR, Src, and Akt by treatment with cixutumumab.

FIG. 23 shows increased recruitment of Src and FAK to integrin β3 by IGF-1 stimulation.

FIG. 24 shows decreased cell adhesion to IGF-1-coated plate by knockdown of IGF-1R or integrin β3 using siRNA transfection.

FIG. 25 shows the integrin β3 binding to IGF-1-coated plate by cixutumumab treatment.

FIG. 26 shows (a) when expression of integrins β1 and β3 are compared under a general 2D culture condition and a 3D-mimetic 2D culture condition using a polyHEMA-coated plate, the β1 expression is decreased in the 3D organoid culture condition, but the β3 expression does not have any difference between the 2D and 3D conditions; (b) the association of integrin β3 expression level with cixutumumab sensitivity.

FIG. 27 shows inhibition of Src activation and concurrent phosphorylation of EGFR and Akt by blockade of integrin β3 with a neutralizing antibody in LN686, FADU and OSC19 cells.

FIG. 28 shows inhibition of phosphorylation of Src, EGFR (Y845), and, to a lesser extent, Akt by treatment with a Src-family kinase (SFK) inhibitor PP2 in LN686, FADU, and OSC19 cells.

FIG. 29 shows decrease of cixutumumab-mediated re-phosphorylation of Src, FAK, EGFR, Aid, and mTOR by combined treatment with an integrin β3 neutralizing antibody (αβ3) or a Src inhibitor (PP2) in LN686 and FADU cells.

FIG. 30 shows no effect on the cixutumumab-mediated re-phosphorylation of Src, FAK, EGFR, Akt, and mTOR by combined treatment with an integrin β1 neutralizing antibody (αβ1) in LN686 and FADU cells.

FIG. 31 shows significant potentiation of growth-inhibitory effect of cixutumumab by combined treatment with an integrin β3 neutralizing antibody (αβ3) or a Src inhibitor (PP2).

FIG. 32 shows inhibition of cixutumumab-mediated re-phosphorylation of Src, FAK, EGFR, Aid, and mTOR by knockdown of Src with siRNA transfection in LN686 and FADU cells.

FIG. 33 shows significant enhancement of growth-inhibitory effect of cixutumumab by knockdown of Src with siRNA transfection in LN686 and FADU cells.

FIG. 34 shows significantly reduced tumor growth by combined treatment with cixutumumab and adenoviruses expressing C-terminal Src kinase (CSK) in LN686 tumor xenografts.

FIG. 35 shows significantly reduced tumor weight by combined treatment with cixutumumab and adenoviruses expressing C-terminal Src kinase (CSK) in LN686 tumor xenografts.

FIG. 36 shows significant enhancement of growth-inhibitory effect of cixutumumab by blockade of integrin β3 using siRNA, or combined treatment with an integrin β1 neutralizing antibody (αβ1) or an inhibitor (PP2).

FIG. 37 shows increase of caspase-3 activation, as demonstrated by increase of caspase-3 activity and cleaved caspase-3 expression, by combined treatment with cixutumumab and an integrin β3 neutralizing antibody (αβ3).

FIG. 38 shows enhanced antitumor effect by combined administration with cixutumumab and an intergrin β3 siRNA in tumor xenograft model using LN686 cells.

FIG. 39 shows stimulation of Src phosphorylation, integrin β3 expression, and an increase in apoptosis when integrin β3 siRNA is administered alone or in combination with cixutumumab in a tumor xenograft model using LN686 cells.

FIG. 40 shows enhanced antitumor effect by combined administration with cixutumumab and SFK inhibitor dasatinib in tumor xenograft using LN686 cells.

FIG. 41 shows stimulation of Src phosphorylation, integrin β3 expression, and an increase in apoptosis when the SFK inhibitor, that is, dasatinib is administered alone or in combination with cixutumumab in a tumor xenograft model using LN686 cells.

FIG. 42 is a schematic diagram of primary resistance to anti-IGF-1R mAbs—primary resistance to anti-IGF-1R mAbs is mediated by alternative proliferative and survival signals through interaction between IGF-1 and integrin β3 and resulting integrin β3-mediated signaling activation when IGF-1R is inhibited by cixutumumab; targeting integrin β3 or Src overcomes resistance to anti-IGF-1R mAbs.

FIG. 43 shows increased phosphorylation of Src and FAK by cixutumumab treatment with IGF-1 stimulation; suppression of these cixutumumab-induced phosphorylation by blockade of IGF-1 and integrin β3 neutralizing antibodies.

FIG. 44 shows attenuation of cixutumumab-induced Src and FAK phosphorylation by transfection with mutant integrin β3, in which the specificity loop of integrin β3 critical for IGF-1 binding (CYDMKTTC, residues 177-184) is replaced with the corresponding sequence of integrin β1 (CTSEQNC, residues 187-193).

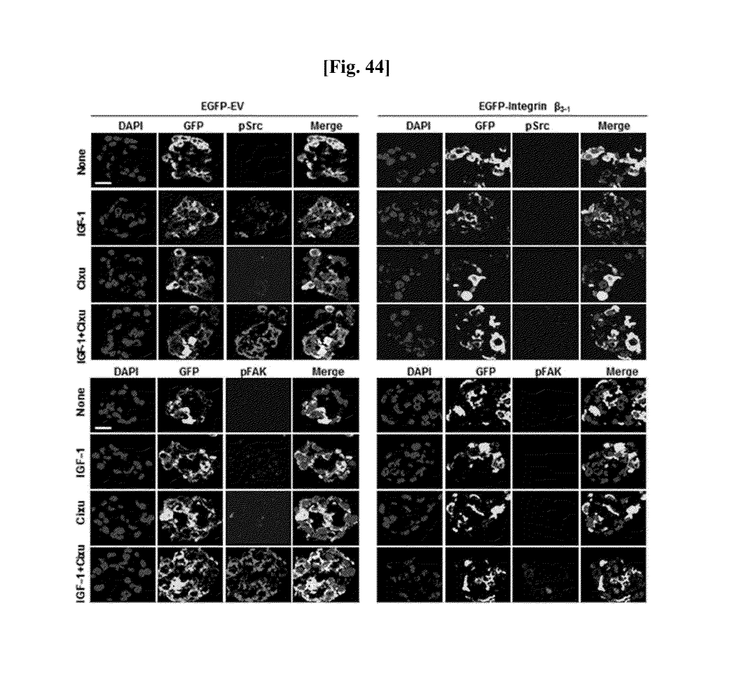

FIG. 45 shows increased Src phosphorylation by cixutumumab treatment in cells attached to extracellular matrix (ECM) in the presence of IGF-1; ablation of such increase by combined treatment with IGF-1 and integrin β3 neutralizing antibodies.

FIG. 46 shows increased FAK phosphorylation by cixutumumab treatment in cells attached to extracellular matrix (ECM) in the presence of IGF-1; ablation of such increase by combined treatment with IGF-1 or integrin β3 neutralizing antibodies.

### BEST MODES OF INVENTION

Based on results of the study, the present inventor suggest a hypothesis in which, if IGF-1 directly binds to integrin β3 and tumors co-express IGF-1R and integrin β3, IGF-1 which fails to bind to IGF-1R due to the presence of IGF-1R mAbs activates signal transduction via alternative binding to integrin β3, mediating resistance to anti-IGF-1R mAbs-based therapies. According to this hypothesis, the inventor completed this invention.

Cells used in Examples of the present invention were maintained in 10% FBS-supplemented RPMI 1640 or DMEM (Life Technologies, Gaitheburg, Md.). Primary tumor specimens were collected from an untreated patient who underwent lobectomies of squamous carcinoma of the oral cavity in the MD Anderson Cancer Center.

Human HNSCC cell lines (UMSCC1, UMSCC2, UMSCC4, UMSCC6, UMSCC11A, UMSCC14A, and UMSCC38) were provided by Dr. T. Carey (University of Michigan, Ann Arbor, Mich.), and LN686, FADU, TR146, HN30, and OSC19 cells were provided by Dr. Jeffrey Myers (MD Anderson Cancer Center, Houston, Tex.). SqCC/Y1 cells were kindly provided by Dr. M. Reiss (Yale University, New Haven, Conn.). H226B and H226Br NSCLC cell lines were provided by Dr. Jack Roth (MD Anderson Cancer Center). Human NSCLC (H596, H460, H1299, A549, and H358) cell lines were purchased from American Type Culture Collection (Manassas, Va.). Athymic nude mice were purchased from Harlan Sprague Dawley (Indianapolis, Ind.). Human recombinant IGF-I, IGF-II, EGF, IGF-1R (Glu31-Asp741), and IGF-1 neutralizing antibodies were purchased from R&D Systems (Minneapolis, Minn.). A humanized mAbs targeting IGF-1R (cixutumumab) and EGFR (Cetuximab, Erbitux) were provided by ImClone Systems (New York, N.Y.). Dasatinib was provided by a pharmacy of MD Anderson Cancer Center. A Src inhibitor (PP2) and an IGF-IR TKI (AG1024) were purchased from Calbiochem-Novabiochem (Alexandria, New South Wales, Australia). A recombinant integrin β3 protein was provided by Dr. Yoko K. Takada (University of California Davis School of Medicine). Neutralizing antibodies against integrin β1 (AIIB2) and integrin β3 (B3A) were purchased from Millipore (Temecula, Calif.). Other materials unless otherwise indicated were purchased from Sigma-Aldrich (St. Louis, Mo.).

A pharmaceutical composition of the present invention may include a pharmaceutically acceptable carrier. The pharmaceutically acceptable carrier may include, but are not limited to, a saline, polyethyleneglycol, ethanol, vegetable oil, and isopropyl myristate. In addition, it is obvious to those of ordinary skill in the art that a dose of the pharmaceutical composition can be widely adjusted according to a patient's weight, age, gender, state of health, diet, administration time and method, an excretion rate, and severity of a disease. As an exemplary embodiment of the present invention, the pharmaceutical composition may be administered at the dose of 0.001 to 100 mg/wt kg, and preferably, 0.01 to 30 mg/wt kg per day.

In the present invention, the “individuals” mean objects to be necessary for treating a disease, and specifically, mean human or non-human primates, and mammals including mice, rats, dogs, cats, horses, and cows.

Hereinafter, exemplary examples will be provided to help understanding the present invention. While the following examples are merely provided to more easily understand the present invention, the scope of the present invention is not limited to the following examples.

### EXAMPLES

### Cell Proliferation

Cells were cultured in 96-well poly(poly-2-hydroxyethyl methacrylate [HEMA])-coated plates (PCPs) or ultra-low attached plates (UAPs). Control IgG1 (25 μg/ml), cixutumumab (25 μg/ml), PP2 (10 μM), and an integrin β3 neutralizing antibody B3A (10 μg/ml) were diluted in culture media containing 10% FBS or IGF-1 and treated alone or in combination. To examine the effect on cancer cell proliferation, a 3-(4,5-dimethylthiazol-2-yl)-2,5-diphenyltetrazolium bromide (MTT) or 3-(4,5-dimethylthiazol-2-yl)-5-(3-carboxymethoxyphenyl)-2-(4-sulfophenyl)-2H-tetrazolium (MTS) assay was performed, and to evaluate the effect on anchorage-independent colony formation, a soft agar colony formation assay was performed according to a method in our previous report (Morgillo et al., 2006).

The PCPs were prepared according to a previously reported method (Fukazawa et al., 1995). In brief, tissue culture plates were collated twice with poly-(HEMA) (10 mg/ml in 95% ethanol) at 37° C. and then washed with phosphate-buffered saline (PBS) several times.

For experiments using viruses, cells were infected with 10 particle forming units (pfus) per cell for Ad-EV or Ad-CSK in serum-free medium for 2 h. After 7 days of incubation, cell proliferation was measured with the MTT and MTS assays. 6 replicated wells were used for each analysis and at least three independent experiments were performed.

To evaluate the effect on anchorage-independent colony formation, cells were suspended in 0.4% agar in media at a density of 2×10³ cells per well, replated in 12-well plates that have been pre-coated with 0.8% agar, and cultured in growth medium containing cixutumumab (25 μg/ml) for 2-6 weeks. Cells were stained with 0.1% Coomassie brilliant blue in PBS, and the colonies >0.2 mm in diameter were counted. Independent experiments were repeated three times.

Western Blot Analysis and Immunoprecipitation

Preparation of total cell lysates, Western blot analysis, and immunoprecipitation were performed according to methods described in our previous report (Morgillo et al., 2006).

Total cell lysates were immunoprecipitated with antibodies against integrin β3, integrin β1, or IgG. The precipitates with protein A agarose were resolved by sodium dodecyl sulfate-polyacrylamide gel electrophoresis (SDS-PAGE) and transferred to a polyvinylidene difluoride (PVDF) membrane. Western blot analysis was performed using specific Abs described as follows, and antigen-antibody complexes were visualized using enhanced chemiluminescence plus kits (Amersham Biosciences, Piscataway, N.J.).

(Antibodies against phospho-Src (Y416), Src, phospho-Akt (S473), Akt, phospho-IGF-1R (Y1131), phospho-mTOR (S2248), mTOR, phospho-p70S6K (T389), p70S6K, phospho-S6K (S235/236), S6K, phospho-4EBβ1 (T37/46), 4EBβ1, phospho-EGFR (Y845), phospho-EGFR (Y1068), and EGFR were purchased from Cell Signaling Technology (Danvers, Mich.); antibodies against IGF-1R3 (C20), phospho-ERK (T202/204), ERK, phospho-Src (Y416), Src, PI3-kinase p85α, Csk (C-20), His (H-15) were purchased from Santa Cruz Biotechnology (Santa Cruz, Calif.); antibodies against phospho-FAK (Y861), integrin β1, integrin β3, and FAK were purchased from BD Biosciences (San Jose, Calif.))

Animal Experiments

All animal experiments were performed according to protocols approved by the Institutional Animal Care and Use Committee of Seoul National University and MD Anderson Cancer Center.

LN686, UMSCC38, H226B, or A549m cells (1×10⁶ cells/mouse in 100 μl of PBS) were subcutaneously injected into nude mice at a single dorsal flank site (6-9 mice per group). For HNSCC patient-derived tumor xenografts, primary human tumor specimens were collected from an untreated patient who underwent lobectomies of squamous carcinoma of the oral cavity. The tumors were cut into small pieces with a size of 2-mm³ and subcutaneously implanted into the nude mice (one piece per mouse). At 0 day (the time point when tumor size reached to 80 to 100 mm³) mice were treated with cixutumumab (10 mg/kg, i.p., once or twice a week), Ad-EV (3×10¹¹ pfu, intratumoral injection, once a week), Ad-CSK (3×10¹¹ pfu, intratumoral injection, once a week), siCon, siintegrin β3 (5 mg/mouse, i.p., once a week), dasatinib (20 mg/kg, oral gavage, daily), cixutumumab and Ad-CSK, or cixutumumab and dasatinib.

Liposomal Preparation

Liposomal formulations were 1,2-dimyristoyl-sn-glycero-3-phosphocholine/1,2-dimyristoyl-sn-glycero-3-[phospho-rac-(1-glycerol)] (sodium salt) or a pegylated version of 1,2-dimyristoyl-sn-glycero-3-phosphocholine/cholesterol/1,2-dimyristoyl-sn-glycero-3-phosphoethanolamine-N-[methoxy-(polyethylene glycol)-2000]. The lipid formulations were dissolved in tert-butanol and filtered using a 0.22-mm filter for sterilization. A glass bottle containing the liposome solution was frozen in a dry ice-acetone container, and then the lipid solution was lyophilized to remove the tert-butanol. The glass bottle was stored at −20° C. and thawed at room temperature before use.

Enzyme-Linked Immunosorbent Assay (ELISA)

IGF-1 (1 or 2 μg) was coated onto wells in 96-well microtiter plates and incubated with recombinant integrin β3 (0.5 μg/ml) and/or IGF-1R (0.1 or 0.5 μg/ml) protein diluted in 1 mM MnCl₂-added HEPES-Tyrode buffer at room temperature for 2 h in the presence or absence of cixutumumab (10 μg/ml). Binding of integrin β3 and/or IGF-1R was detected by enzyme immunoassay using anti-His or anti-IGF-1R mAbs and avidin-labeled alkaline phosphatase-conjugated anti-mouse or anti-rabbit IgGs.

Statistical Analysis

Data obtained from the MTT and MTS assays were analyzed using Student t test. All sample means and 95% confidence intervals (CIs) from multiple samples (n=5-8) were calculated using Microsoft Excel 2007 software (Microsoft Corporation, Seattle, Wash.). The statistical significance of differences in tumor growth in the combination treated group and in the single treatment group were analyzed using the one-way analysis of variance (IBM SPSS version 21, Armonk, N.Y.). In all statistical analyses, two-sided P values of <0.05 were considered statistically significant.

### Example 1

### Evaluation of the Effect of Cixutumumab, an Anti-IGF-1R mAb, on Growth of HNSCC and NSCLC Cells in Conventional 2D Culture Conditions

A panel of 13 HNSCC and 6 NSCLC cells grown in 2D culture conditions using TCPs were treated with 25 μg/ml cixutumumab, and the effect of cixutumumab on growth of cancer cells tested was analyzed by the MTT assay.

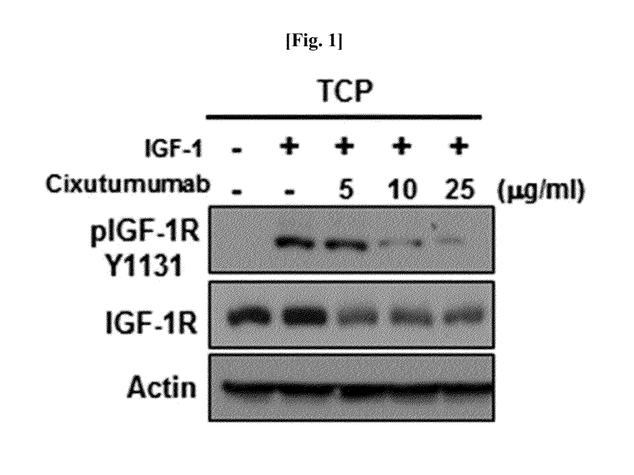

LN686 cells were treated with increasing concentrations of cixutumumab for 6 h, and then stimulated with 100 ng/ml IGF-1 for 30 min. Regulation of pIGF-1R (Y1131) and IGF-1R expression by cixutumumab is shown in FIG. 1.

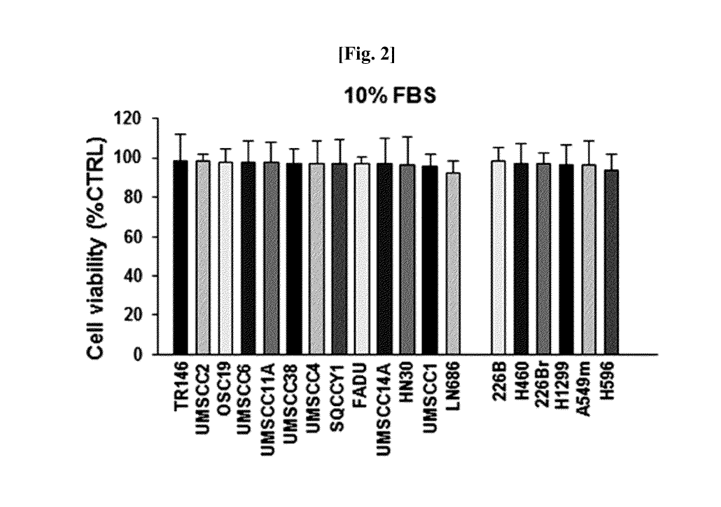

The effect of cixutumumab on growth of 13 HNSCC and 6 NSCLC cells was evaluated by the MTT assay, and the results are shown in FIG. 2.

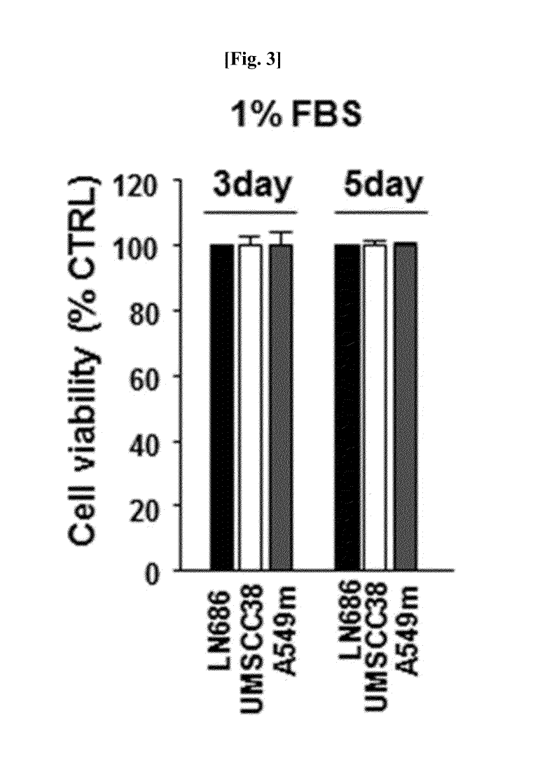

LN686, UMSCC38, and A549m cells were treated with cixutumumab diluted in 1% FBS-containing medium for 3 and 5 days. Cell viability was evaluated by the MTT assay. The results are shown in FIG. 3.

As shown in FIGS. 1 to 3, IGF-1-induced IGF-1R phosphorylation was markedly inhibited by treatment with cixutumumab; however, cixutumumab did not inhibit the viability of cells grown in 2D culture conditions for up to 7 days. In addition, there is no difference in the effect of cixutumumab between normoxic and hypoxic conditions (data not shown), suggesting serum concentrations or oxygen levels had no influence on the effect of cixutumumab on cell viability in 2D.

### Example 2

### Effect of Cixutumumab on Growth of HNSCC and NSCLC Cells Under Reduced Cell Adhesion and Anchorage-Independent Culture Conditions

It is known that the growth, response to signal transduction, and effect of drug treatment in cells grown in 3D conditions are similar to in vivo but largely different from those of cells grown in vitro in TCPs (Mizushima et al., 2009a). Therefore, the present inventor evaluated the effect of cixutumumab using poly-2-hydroxyethyl methacrylate (polyHEMA)-coated plates (PCPs) and ultra-low attached plates (UAPs). These 2D culture conditions mimic 3D conditions through suppression of cell adhesion and spreading (Mizushima et al., 2009a).

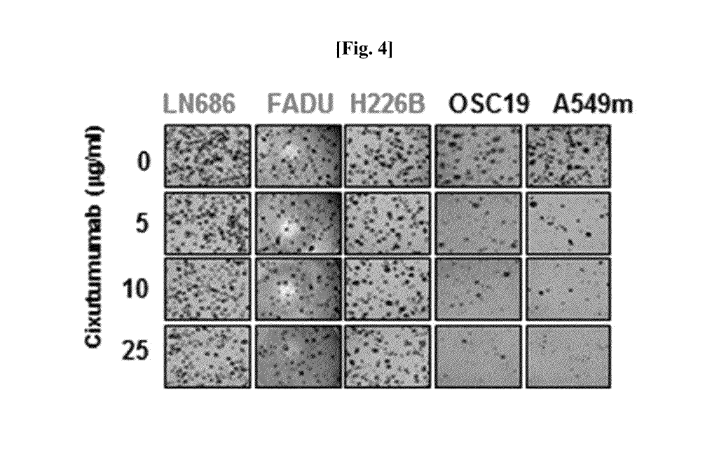

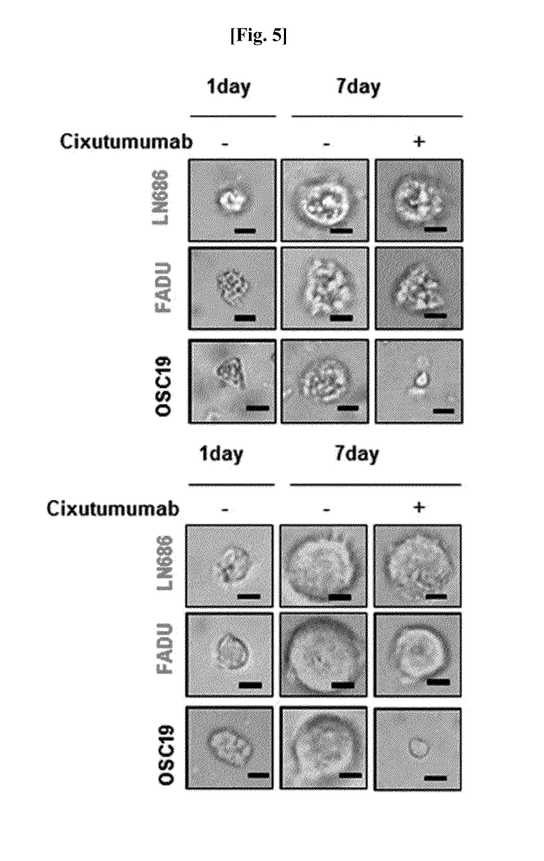

The inhibitory effect of cixutumumab on colony formation of HNSCC and NSCLC cells in soft agar was shown in FIG. 4, and representative single spheroidal colonies of LN686, FADU, and OSC19 cells grown in PCPs or UAPs are shown in FIG. 5.

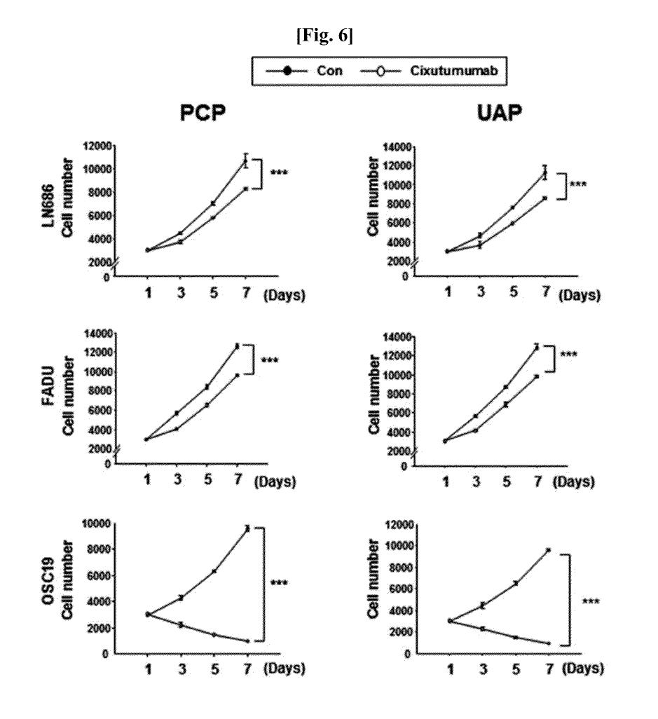

As shown in FIGS. 4 and 5, HNSCC and NSCLC cells formed spheroidal cell masses and grew for at least 7 days in these conditions. Since cell growth rates could be different under each set of culture conditions, the present inventor counted the cells at distinct culture periods and depicted the results in FIG. 6. The values were represented as mean±standard deviation (SD), and a statistical significance was evaluated using two-sided Student t test (*P<0.05; **P<0.01).

As shown in FIG. 6, there was no difference in growth rates of LN686 and FADU cells between control cells and those treated with cixutumumab. In contrast, cixutumumab-treated OSC19 cells exhibited significantly slower growth rates than control cells.

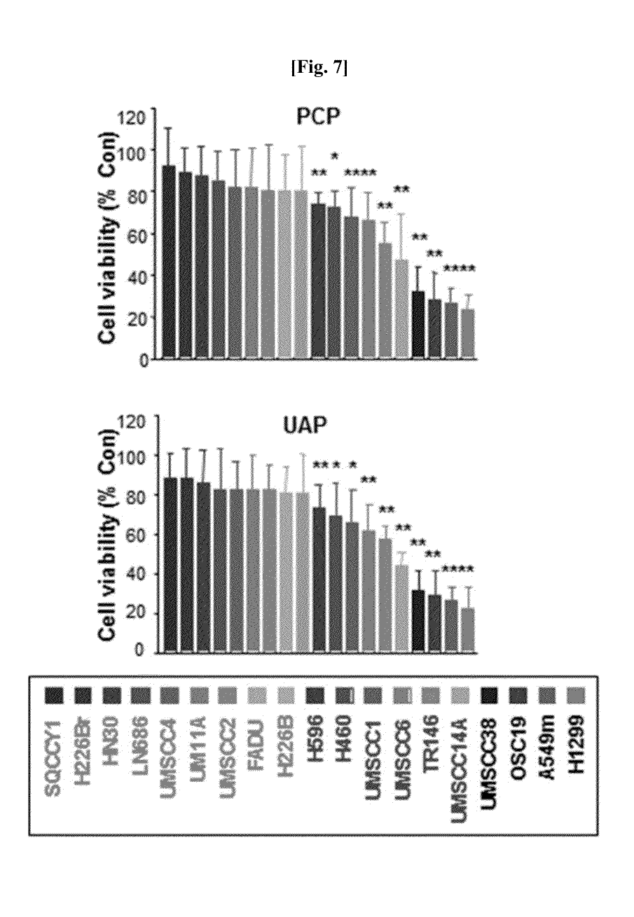

According to these results, the present inventor examined the sensitivity of 13 HNSCC and 6 NSCLC cells to cixutumumab treatment using the 3-(4,5-dimethylthiazol-2-yl)-5-(3-carboxymethoxyphenyl)-2-(4-sulfophenyl)-2H-tetrazolium (MTS) assay. HNSCC and NSCLC cells grown in PCPs and UAPs were treated with cixutumumab (25 μg/ml) for 7 days. The results are shown in FIG. 7. Each bar represents mean±SD of 6 wells of a single representative experiment.

As shown in FIG. 7, after treatment with cixutumumab in PCPs, 7 HNSCC and 2 NSCLC cell lines (red) experienced less than 20% inhibition in viability, 4 HNSCC and 2 NSCLC cell lines (blue) experienced 20% to 50% inhibition in viability, and the remaining 2 HNSCC and 2 NSCLC cell lines (black) experienced more than 60% inhibition in viability. Therefore, the present inventor defined the red lines as “resistant”, blue lines as “mildly sensitive,” and black lines as “sensitive.” These cell lines grown in UAPs had similar responses to cixutumumab treatment compared to those grown in PCPs.

### Example 3

### Effect of Cixutumumab on Growth of Cells Grown in In Vitro 3D Conditions and In Vivo

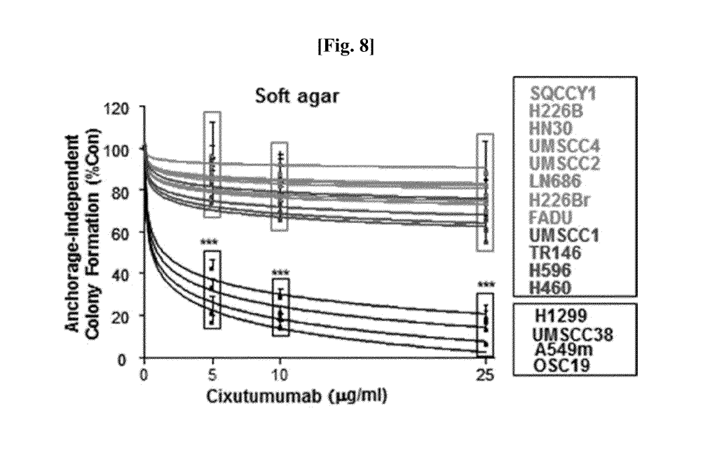

Because cells grown in the 3D environment might differ from cells growing in PCPs and UAPs, the present inventor evaluated the effects of cixutumumab on cell growth in soft agar, a 3D culture system. 10 HNSCC and 6 NSCLC cells were cultured in soft agar for 4 to 6 weeks in the presence or absence of cixutumumab (25 μg/ml). The results are shown in FIG. 8

After cixutumumab treatment, all of the NSCLC and HNSCC cell lines that were sensitive to drug treatment in PCPs and UAPs formed significantly fewer colonies in soft agar than did the resistant and mildly sensitive lines (FIG. 8). None of these cells grown in PCPs, UAPs, or soft agar showed significant decrease in proliferation after treatment with 25 μg/ml IgG (data not shown).

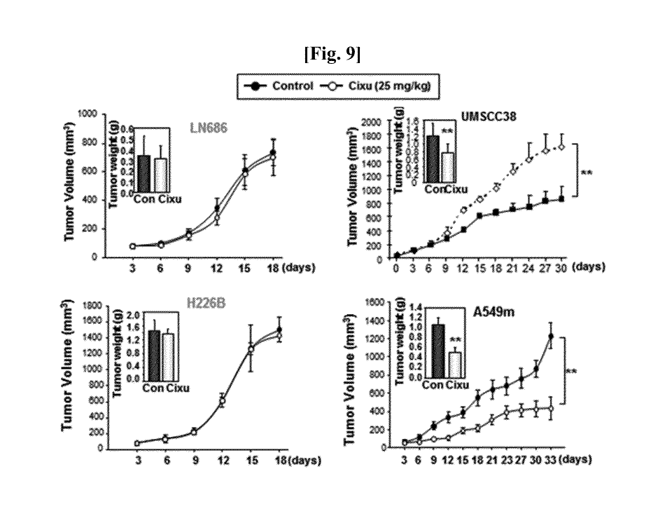

Finally, the present inventor examined the effect of cixutumumab in vivo by treating mice harboring xenograft tumors of the drug-resistant (LN686 and H226B) and -sensitive (UMSCC38 and A549m) cells for 18 to 33 days until control mice showed necrotic tumors or tumors ≧1.5 cm in diameter. The results are shown in FIG. 9. Tumor xenografts were treated with vehicle or cituxumumab (intraperitoneal, 25 mg/kg) twice a week. Data are presented as mean tumor volume ±SD for indicated time or mean tumor weight at the date of euthanasia ±SD (*P<0.05, **P<0.01, ***P<0.001). As shown in FIG. 9, cixutumumab significantly decreased the growth and weight of UMSCC38 and A549 xenograft tumors, whereas no significant effects were observed in LN686 and H226B xenografts. Thus, antitumor activity of cixutumumab was consistent in cells grown in 3D (or 3D-mimicking) conditions in vitro and in those grown in vivo.

### Example 4

### Analysis of Mechanism of Resistance to Anti-IGF-1R Monoclonal Antibody

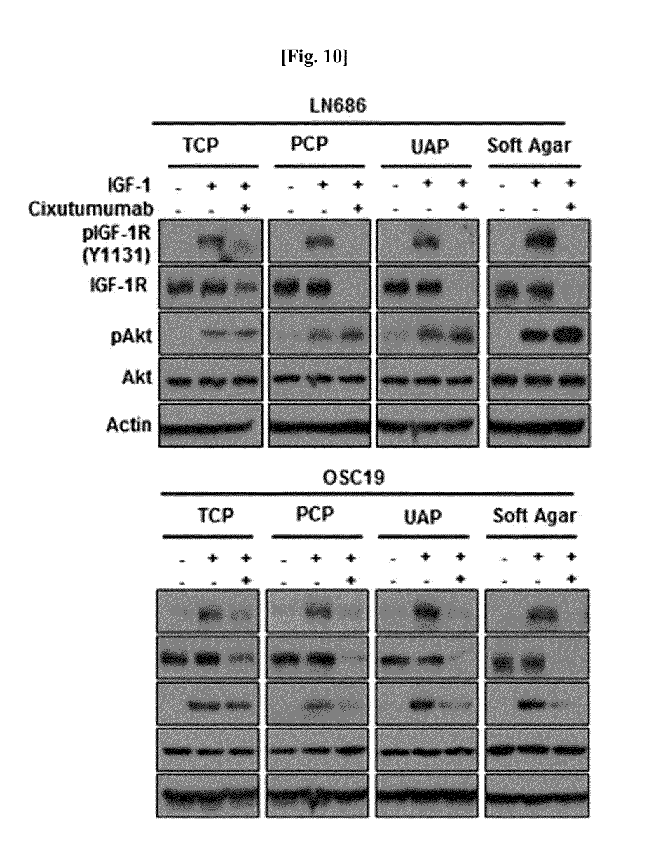

The present inventors investigated the mechanisms involved in the resistance to cixutumumab treatment in HNSCC and NSCLC cells. cixutumumab resistant (LN686, FADU, 226B, 226Br, H596, and H460) or -sensitivity (UMSCC38, OSC19, H1299, and A549m) lines grown in TCP, PCP, UAP, or soft agar were untreated or treated with 25 μg/ml of cixutumumab for 6 h, and then were stimulated with IGF-1 for 30 min. Expression of several proteins were analyzed by Western blot analysis. The results are shown in FIG. 10.

As shown in FIG. 10, the present inventors first assessed the function of cixutumumab in IGF-1R signaling in cixutumumab-resistant and sensitive cell lines grown in PCPs. Cixutumumab treatment (25 μg/mL for 6 h) effectively suppressed IGF-1-induced IGF-1R and Akt phosphorylation, and total IGF-1R expression in both cixutumumab-resistant LN686 and cixutumumab-sensitive OSC19 cells grown in TCPs, PCPs, UAPs, as well as in soft agar. Whereas in the case of OSC19 sensitive to cixutumumab, phosphorylation of Aid, and its downstreameffectors, including mTOR, p70S6K, and S6 were inhibited by cixutumumab, but LN686 resistant to cixutumumab showed a tendency not to inhibit or to even increase phosphorylation of such kinases.

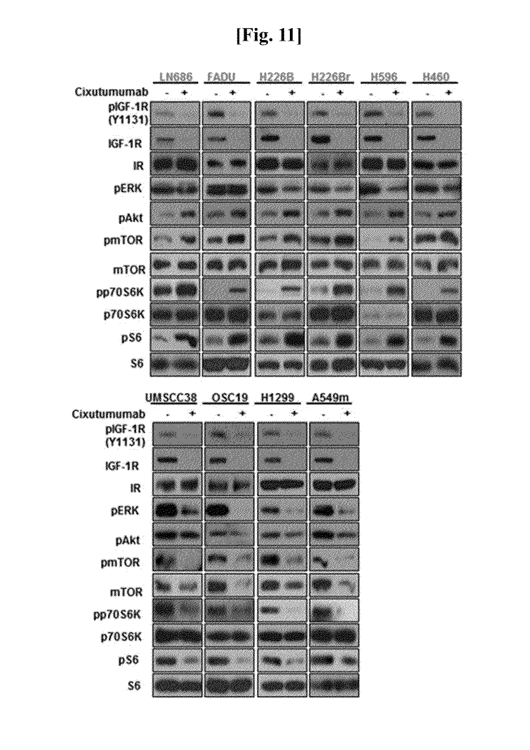

Cixutumumab-resistant (LN686, FADU, 226B, 226Br, H596, and H460) or -sensitive (UMSCC38, OSC19, H1299, and A549m) cell lines grown in PCP were untreated or treated with 25 μg/ml of cixutumumab for 7 days, and then were stimulated with 10% FBS for 30 min. Expression of several proteins were analyzed by western blot analysis. The results are shown in FIG. 11.

ERK1/2 phosphorylation did not show any difference in sensitivity to cixutumumab. Such a result implied that a resistance mechanism of cixutumumab is involved with activation of Aid and its downstream effectors, and therefore, to investigate the mechanism, an effect of activation of another signal transduction capable of activating Aid, mTOR, etc. in addition to IGF-1R was confirmed. Previous studies have suggested that expression of insulin receptor (IR) is implicated in resistance anti-IGF-1R mAbs (Cao et al., 2008; Gong et al., 2009; Huang et al., 2009a; Ulanet et al. 2009; Zha et al., 2010). However, according to the results of this study, as shown in FIG. 11, there was no obvious correlation in expression of IR between cixutumumab-sensitivity or -resistant cellsin before and after the drug treatment. And it is implicated that there is no obvious correlation between IR expression and cixutumumab-sensitivity.

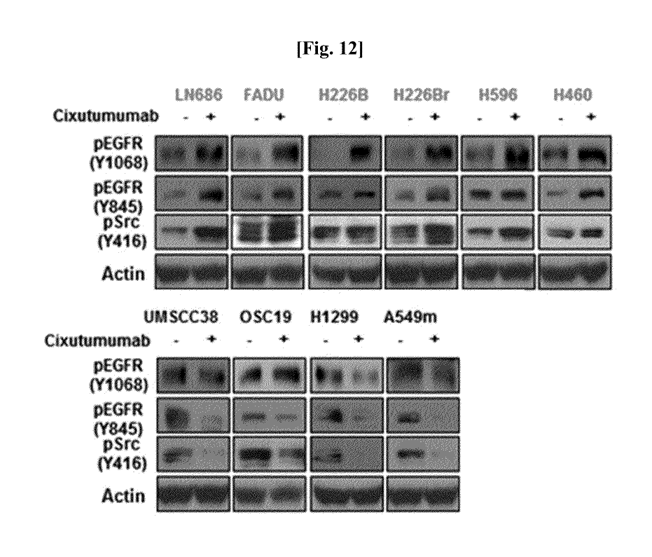

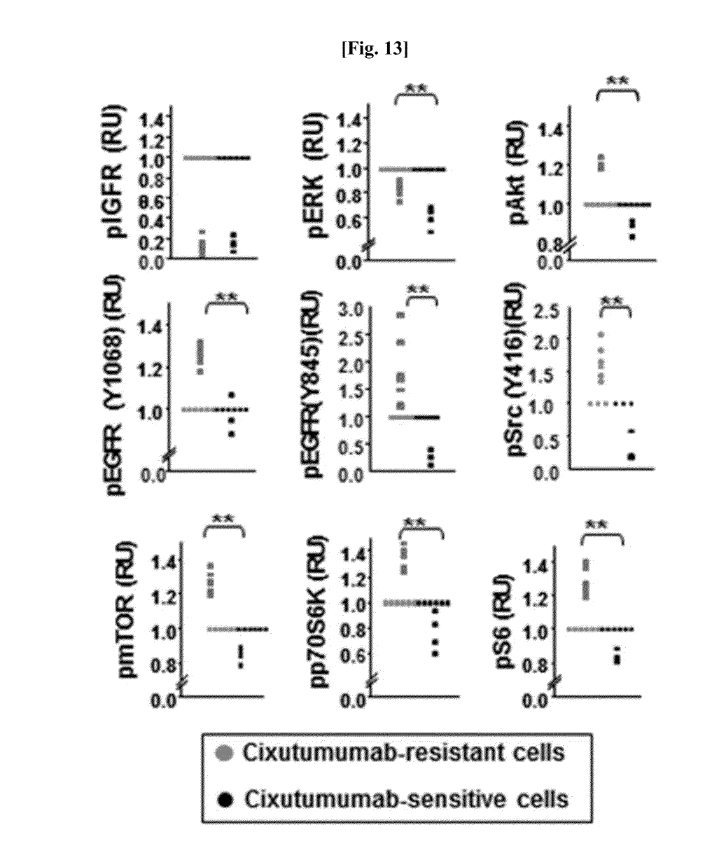

Because cross-talk between EGFR and IGF-1R signalings have been proposed as a major mechanism of a resistance to TKIs of IGF-1R and EGFR (Buck et al., 2008; Morgillo et al., 2006), the present inventors next examined whether cixutumumab treatment induces activation of EGFR and Akt phosphorylation. After cixutumumab-resistant (LN686, FADU, 226B, 226Br, H596, and H460) or -sensitive (UMSCC38, OSC19, H1299, and A549m) cell lines grown in PCPs were treated with cixutumumab, 10% FBS was treated for 30 min. Several phosphorylated proteins and actin were analyzed by western blot analysis. The results are shown in FIG. 12. In addition, densitometric analysis was performed to quantify the expression of the proteins for comparisons between cixutumumab-treated and control cells treated with vehicle. The results are shown in FIG. 13.

As shown in FIGS. 12 and 13, 7 days later the cixutumumab treatment resulted in the increased phosphorylation of EGFR at Tyr1068, an autophosphorylation site, in the drug-resistant lines. Interestingly, cixutumumab treatment also increased phosphorylation of EGFR at the Src-specific site (Y845), along with phosphorylation of Src (Y416), in the cixutumumab-resistant lines. In addition, since Aid, mTOR, p70S6K, S6, EGFR, and Src phosphorylation was increased in the cixutumumab-resistant lines, it was considered that such phosphorylation of signal transduction was significantly correlated with resistance to cixutumumab.

### Example 5

### Mechanism of Cixutumumab-Resistance by Activation of Src and Integrin β3

5-1. Confirmation Using Western Blot Analysis

Stimulation of growth factor receptors, such as EGFR and IGF-1R, increases the activity of c-Src (hereinafter, referred to as Src) (Yeatman, 2004), and Src phosphorylates EGFR and IGF-1R (Maa et al., 1995; Peterson et al., 1996). Both of EGFR and the Src can activate PI3K/Akt pathway, and to assess their contributions to the cixutumumab-induced Akt activation, the present inventors monitored the cixutumumab-induced phosphorylation of EGFR and Src in LN686 cells.

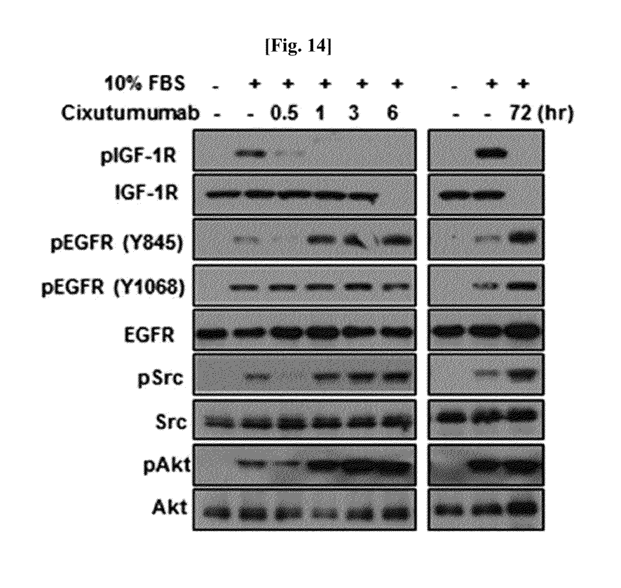

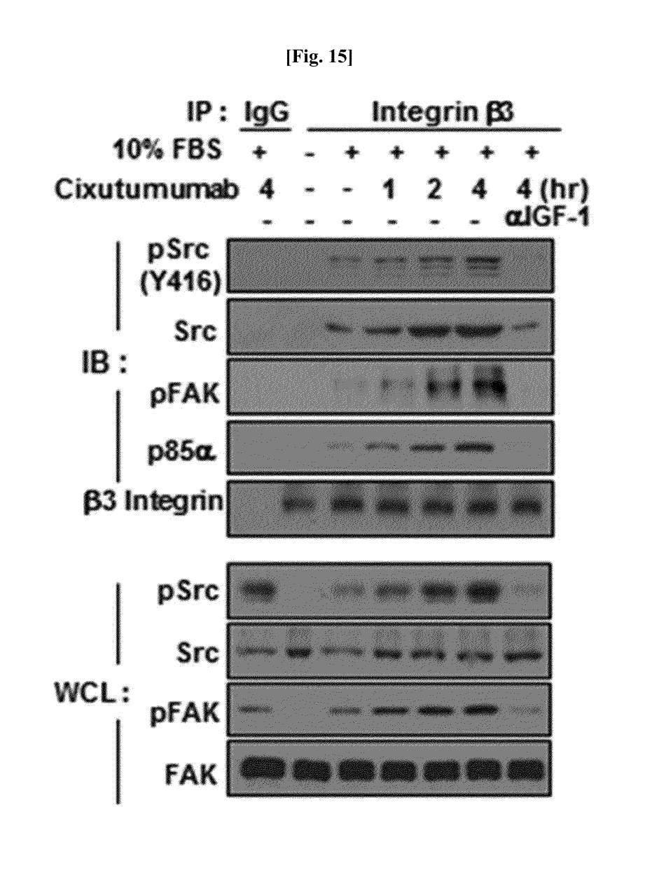

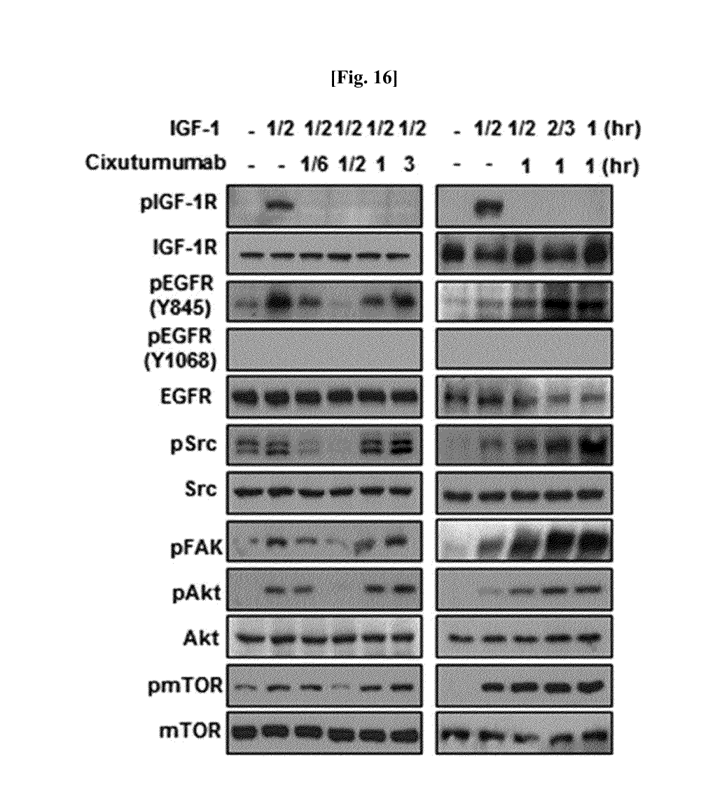

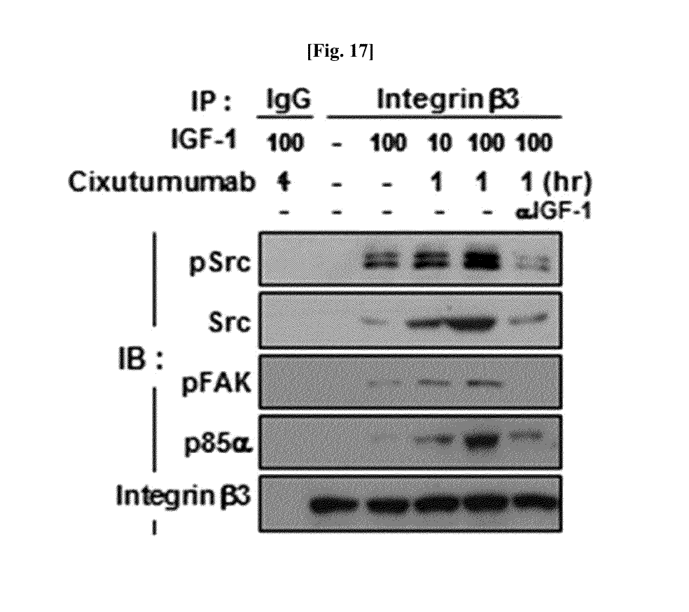

The LN686 cells grown in PCP were treated with cixutumumab (25 μg/ml) for indicated times prior to stimulation with 10% FBS (FIGS. 14 and 15) or IGF-1 (100 ng/ml) (FIGS. 16 and 17) in the presence or absence of anti-human IGF-1 neutralizing monoclonal antibody (αIGF-1) (FIGS. 15 and 16) for indicated times (FIG. 16) or for 30 min (FIGS. 14, 15, and 17). Immunoprecipitation with whole proteins or integrin β3 antibodies was performed on the presented proteins, and expression of the proteins was analyzed by western blot analysis.

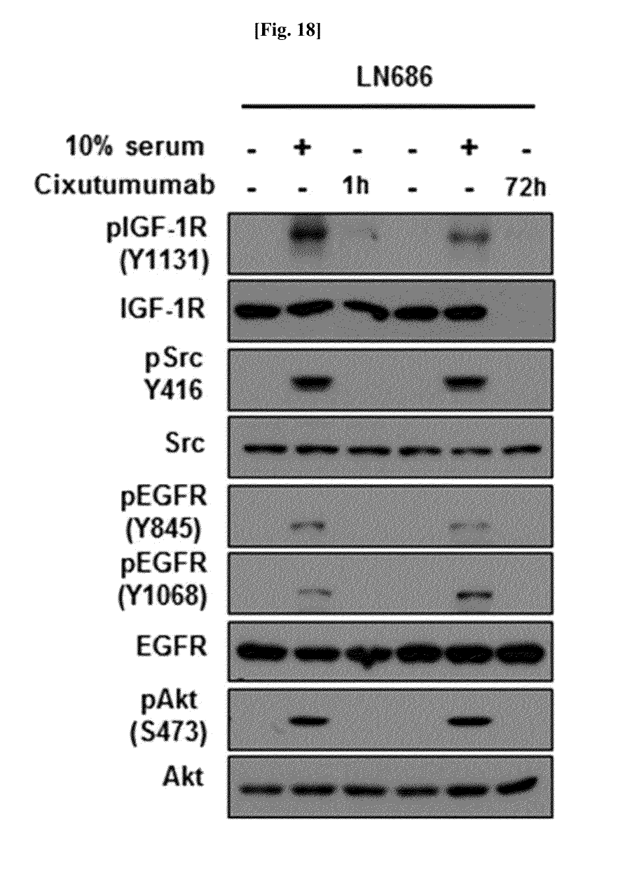

In addition, LN686 cells grown in PCP were treated with cixutumumab (25 μg/ml) for 7 days in the absence of FBS. The results obtained by analyzing p-IGF-1R (Y1131), IGF-1R, p-Src (Y416), Src, p-EGFR (Y845), p-EGFR (Y1068), EGFR, p-Akt (S473), and Akt using western blot analysis. The results are shown in FIG. 18. LN686 cells stimulated with 10% serum for 30 min were included as a control.

As shown in FIG. 14, FBS stimulation (10%, 30 min) induced phosphorylation of IGF-1R, EGFR (Y845, Y1068), Src(Y416), and Akt (S473). Levels of p-IGF-1R, p-EGFR (Y845), p-Src (Y416), and p-Akt (S473) were decreased after 30 min pretreatment with cixutumumab, most likely because of the drug-induced blockade of IGF-1R, and subsequent inactivation of Src and PI3K/Akt.

After cixutumumab treatment, IGF-1R remained dephosphorylated for 3 h with no change in IGF-1R level. Whereas Src, FAK, p-EGFR (Y845), Aid, and mTOR were rapidly rephosphorylated one hour treatment with cixutumumab. IGF-1R levels were decreased after 6 h. p-EGFR (Y1068) and EGFR levels remained unchanged during 6 h of cixutumumab treatment, but increased after 72 h. The kinetics and magnitude of the phosphorylated proteins noted above suggested that blockade of IGF-1R by cixutumumab treatment caused an initial dephosphorylation of Src and its downstream effectors, but other signal transductions were activated, signal transductions of EGFR, Akt, etc. were activated.

It has been reported that several upstream signaling pathways are involved in the activation of Src. Of these, signaling through integrin is known to activate Src (Hood and Cheresh, 2002), and IGF-1 has the ability to directly bind to a specificity loop of integrin β3 (Saegusa et al., 2009). To begin investigating the mechanism of cixutumumab-induced Src activation, the present inventors assessed possible changes in IGF-dependent interaction between integrin β3 and intracellular proteins and activation of its downstream effectors after the cixutumumab treatment.

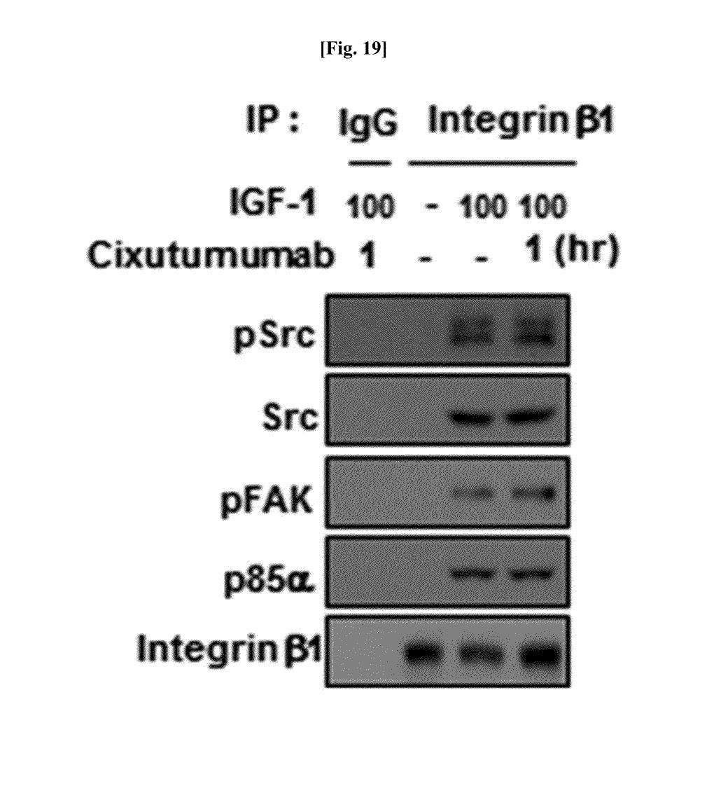

The present inventors confirmed that treatment with cixutumumab enhanced FBS-induced associations between integrin β3 and Src or p85 and concurrent phosphorylation of FAK and Src in a time-dependent manner (FIG. 15). Furthermore, blockade of the IGF-1 actions using IGF-1 neutralizing mAb (αIGF-1) completely abolished the effects of cixutumumab on integrin β3 interaction with intracellular proteins and phosphorylations of Src, FAK, EGFR, Akt, and mTOR. cixutumumab-enhanced integrin β3 signaling was also observed after IGF-1 stimulation (FIG. 16, left). Pretreatment of cixutumumab rapidly blocked the IGF-1-induced phosphorylation of Src and its downstream effectors even after 10 min, however, these proteins were rapidly rephosphorylated after one hour, and it is seen that as the cixutumumab treating time was increased, a degree of rephosphorylation was also increased (FIG. 16, right). Cixutumumab pretreatment also enhanced the IGF-dependent association between integrin β3 and p-Src, p-FAK, or p85α, as shown by the blockade of the association after αIGF-1 treatment (FIG. 17). In contrast, cixutumumab alone failed to induce EGFR, Src, or Akt phosphorylation in the absence of IGF-1 stimulation (FIG. 18). Associations between integrin β1 and p-Src (Y416) or p-FAK (Y861) was also observed after IGF-1 stimulation, however, cixutumumab pretreatment did not affect these associations (FIG. 19, right).

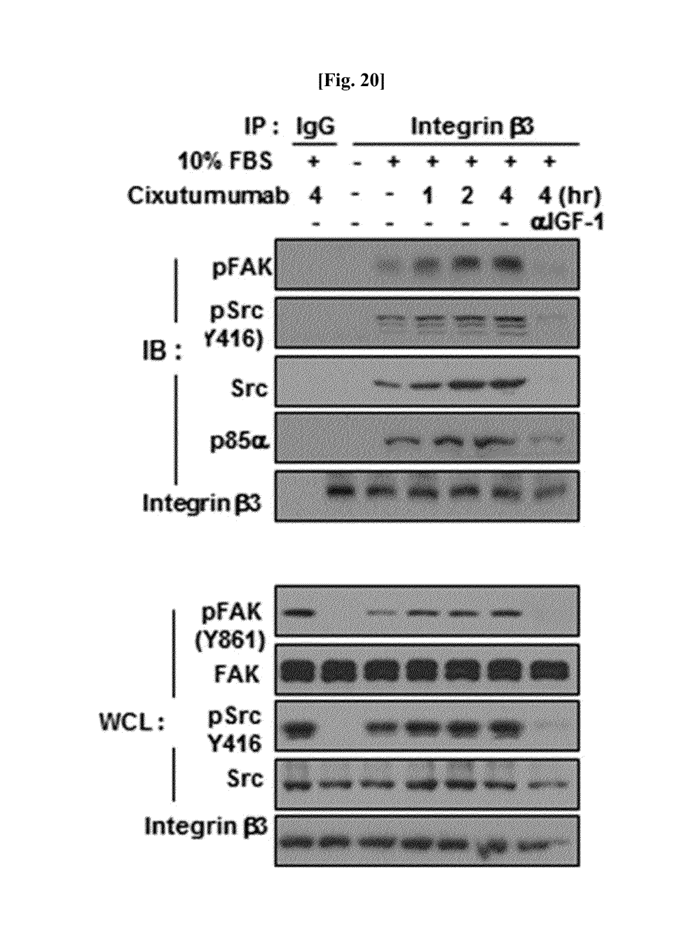

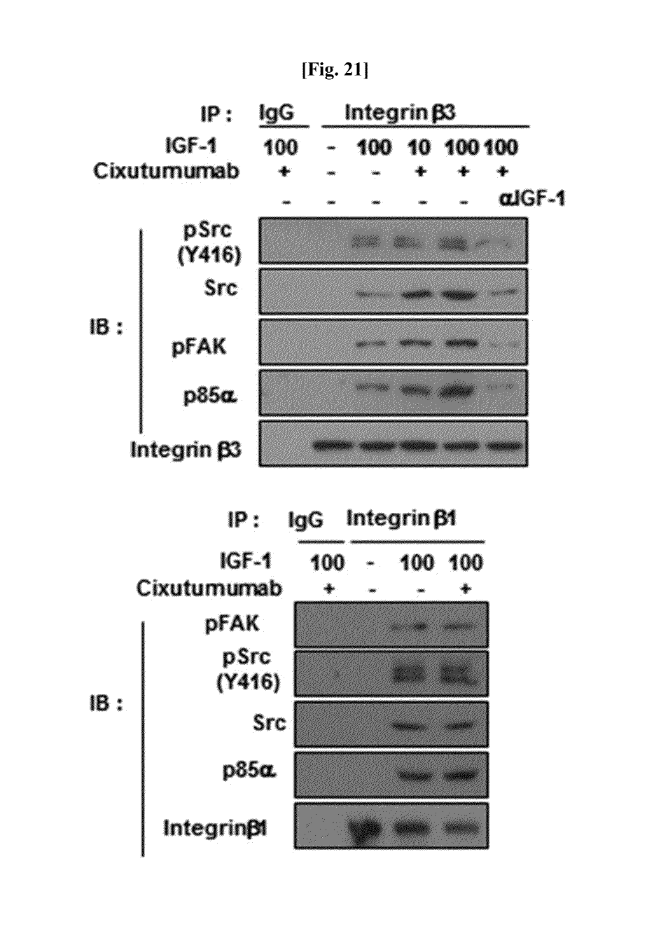

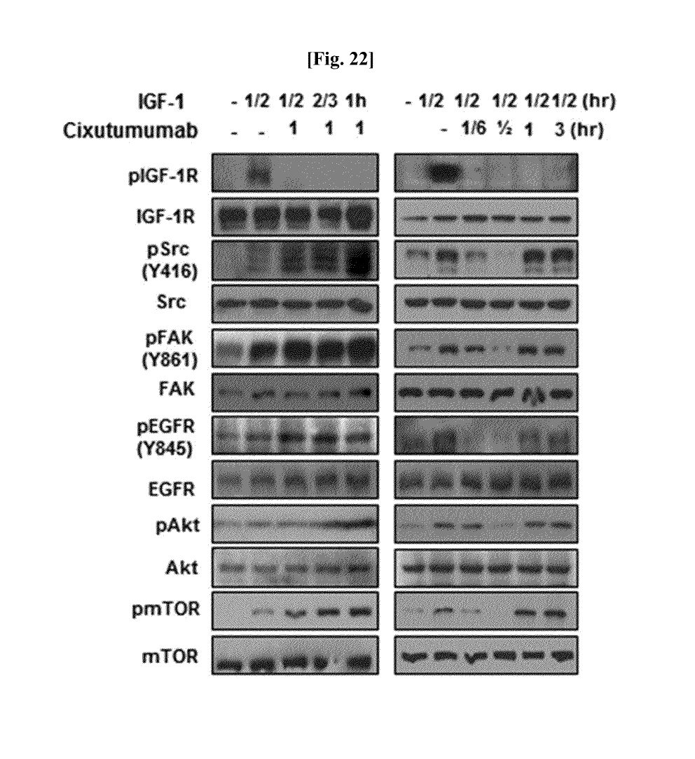

FADU and H226Br cells grown in PCP were treated with cixutumumab (25 μg/ml) for indicated times in the presence or absence of anti-human IGF-1 neutralizing monoclonal antibody (αIGF-1) prior to stimulation with 10% FBS or IGF-1 (100 ng/ml) for 30 min or for indicated time. β3 integrin or β1 integrin immunoprecipitate (IP) and whole cell lysates (WCL) were analyzed by western blot for p-IGF-1R (Y1131), IGF-1R, p-EGFR (Y845), EGFR, p-Src (Y416), Src, p-Akt (S473), and Akt, p-FAK (Y861), p85αsubunit of PI3K, integrin β3 and integrin β1. The results are shown in FIGS. 20 to 23.

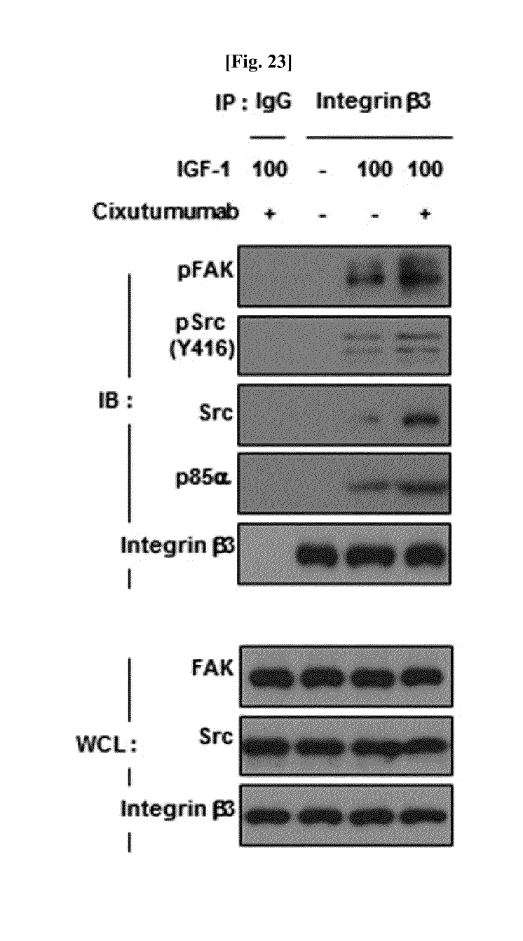

Physical interaction between integrin β3 and Src, phosphorylated Src, p85α, and phosphorylated FAK were also enhanced in the presence of IGF-1 in the FADU (FIGS. 20 to 22) and H226Br (FIG. 23) cells, and EGFR, Akt, Src, mTOR, and FAK phosphorylation was induced by cixutumumab treatment. When the IGF-1 action was neutralized with αIGF-1, a similar tendency to suppress such interaction and phosphorylation was shown.

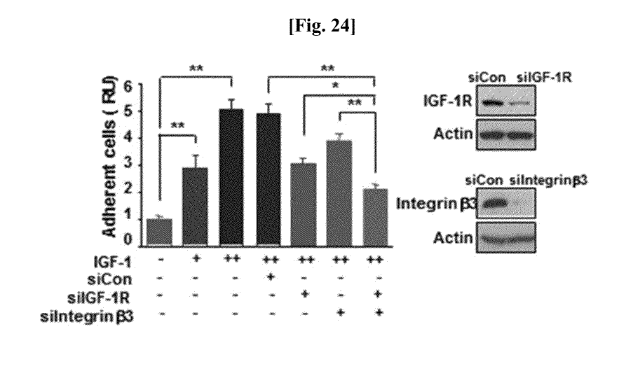

Afterward, the present inventors assessed whether cixutumumab treatment enhances the binding of IGF to integrin β3 by two different binding assays. Firstly, the present inventors prevented silenced expression of IGF-1R, integrin β3, or H226Br cells by transfection with small interfering RNA (siRNA), and analyzed the adherent capacity of the cells to immobilized IGF-1 (Saegusa et al., 2009). Specifically, after IGF-1 was coated onto 96-well microtiter plates at 1(+) or 2(++) mg/ml coating concentrations, adhesion of H226Br cells transfected or untransfected with scrambled siRNA (siCon), IGF-1R-specific or integrin β3-specific siRNA was analyzed by ELISA. Integrin β3 and IGF-1R levels after siRNA treatment were detected by western blot. And the results are shown in FIG. 24.

As shown in FIG. 24, the present inventors found that H226B cells adhered to IGF-1 in a dose-dependent manner, whereas H226Br cells that lost IGF-1R and β3 integrin expression showed reduced adhesion to IGF-1. Cotransfection with IGF-1R and integrin β3 siRNAs suppressed the adhesion of the H226B cells significantly more effectively than did transfection with either siRNA alone.

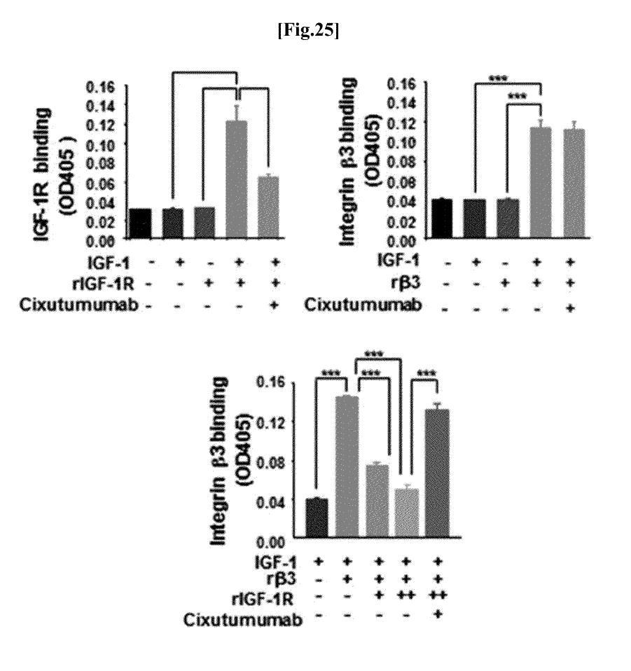

Secondly, the present inventors performed Enzyme-Linked ImmunoSorbent Assay-type integrin binding assay. Specifically, 96-well microtiter plates coated with IGF-1 were recombinant soluble IGF-1R (rIGF-1R; 5 μg/ml) or recombinant soluble integrin β3 (rβ3; 5 μg/ml), alone or in combination, in the presence or absence of cixutumumab (25 μg/ml) for 2 h. And the results are shown in FIG. 25. Bound IGF-1R (left) and integrin β3 (middle, right) were identified using anti-IGF1R and anti-His mAbs, respectively. The data are shown as the mean±SD of triplicate experiments (*P<0.05; **P=0.01; ***P<0.001).

As shown in FIG. 25, Wells of microtiter plates coated with IGF-1 were incubated with recombinant soluble integrin β3 (rβ3), recombinant soluble IGF-1R protein (rIGF-1R), or both in the presence or absence of cixutumumab. The present inventors observed significant binding of both rIGF-1R (FIG. 25, left) and rβ3 (FIG. 25, middle, right) to IGF-1-coated plates. The rβ3 binding to IGF-1-coated plate was suppressed by rIGF-1R in a dose-dependent manner, and such action became clear as an amount of the protein of IGF-1R was increased (FIG. 25, right). Likewise, the ablation of IGF-1-rβ3 interaction by rIGF-1R was markedly blocked by cixutumumab treatment.

5-2. Confirmation Using Fluorescent Immunostaining

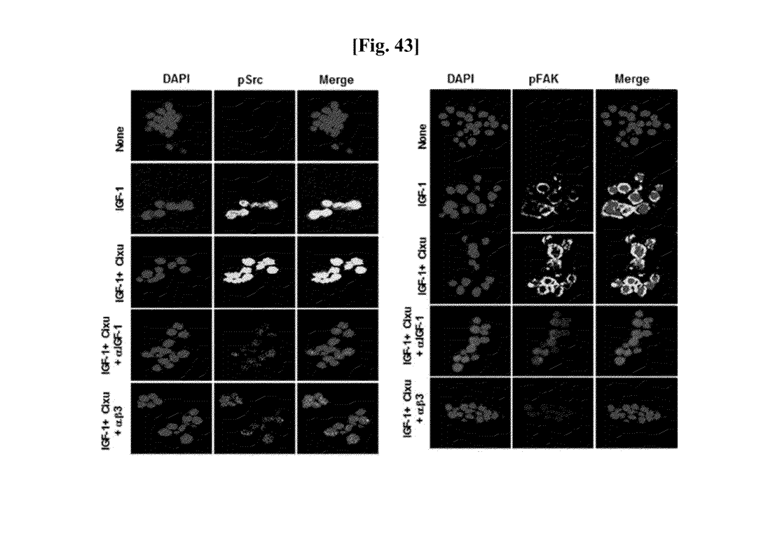

The present inventors reconfirmed the western blot analysis results in which Src and FAK phosphorylation was increased due to cixutumumab in the presence of IGF-1 by fluorescent immunostaining. Cultured LN686 cells in UAPs with cixutumumab alone or in combination with IGF-1 or an integrin β3 neutralizing antibody, treated with IGF-1 to fix, and then Src and FAK phosphorylation was confirmed. As shown in FIG. 43, IGF-1 treatment enhanced Src and FAK phosphorylation, and cixutumumab treatment enhanced phosphorylation. Such an action of the cixutumumab was abolished by the IGF-1 or integrin β3 neutralizing antibody, and therefore it was reconfirmed that the action of cixutumumab for inducing the Src and FAK phosphorylation was mediated by integrin β3 in the presence of IGF-1.

In addition, in the case of cells expressing modified integrin β3, the following experiment was performed to confirm whether or not the Src and FAK phosphorylation was increased by cixutumumab.

According to the Previous study, a modified integrin β3 expression vector was prepared by substituting an amino acid sequence (CYDMKTTC, residues 177-184) of the integrin β3 essential for the IGF-1 bond with an amino acid sequence (CTSEQNC, residues 187-193) of integrin β1 at a corresponding position. An empty vector or a modified integrin β3 expression vector was transfected into FADU cells. These cells were plated, and cultured along with cixutumumab (25 μg/ml) alone or in combination with IGF-1 or an integrin β3 neutralizing antibody for 4 h. After the culture, IGF-1 (100 mg/ml) was treated for 1 hour, and the cells were recovered, washed with PBS, and fixed with 10% formalin for 2 h. When the fixed cells were LN686 cells, they were put into a paraffin block, and when the fixed cells were FADU cells, they were put into an OCT block. Then, the block was divided to form a section having a thickness of 4 μm. In case of the paraffin block, paraffin was removed, and the cells were rehydrogenated by changing alcohol to water. The cells were then washed with PBS and fixed, permeabilized by treating 0.3% Triton-X 100, and cultured with a primary antibody (pSrc or pFAK). The cells were washed with PBS twice, and cultured along with a fluorescent-labeled secondary antibody. After the culture, the cells were washed with PBS, and mounted to observe them using a confocal microscope.

As shown in FIG. 43, it was seen that, unlike an empty vector-transfected cells, Src and FAK phosphorylation caused by cixutumumab was not increased in the presence of IGF-1 in the cells into which a modified integrin β3 expression vector was transfected, and therefore it was confirmed that the Src and FAK phosphorylation caused by cixutumumab was increased by binding the IGF-1 to the integrin β3 since the binding of IGF-1 to IGF-1R was interrupted by cixutumumab.

It is known that when the cells were bound to an extracellular matrix, integrin-mediated signal transduction was activated. Therefore, in the condition in which the Src and FAK phosphorylation caused by integrin was induced by adhering the cells to the extracellular matrix, an effect of cixutumumab on the Src and FAK phosphorylation was confirmed.

A coverslip coated with ECM (collagen or matrigel) was prepared, and the LN686 cells cultured along with cixutumumab alone or in combination of IGF-1 or an integrin β3 neutralizing antibody were adhered to the ECM-coated coverslip in the presence of IGF-1. The adhered cells were fixed with 4% formaldehyde, permeabilized with a Triton X-100 solution, and cultured with a primary antibody (pSrc or pFAK). The cells were washed with PBS twice, and cultured with a fluorescent-labeled secondary antibody. After the culture, the cells were washed with PBS, and mounted to observe them using a confocal microscope.

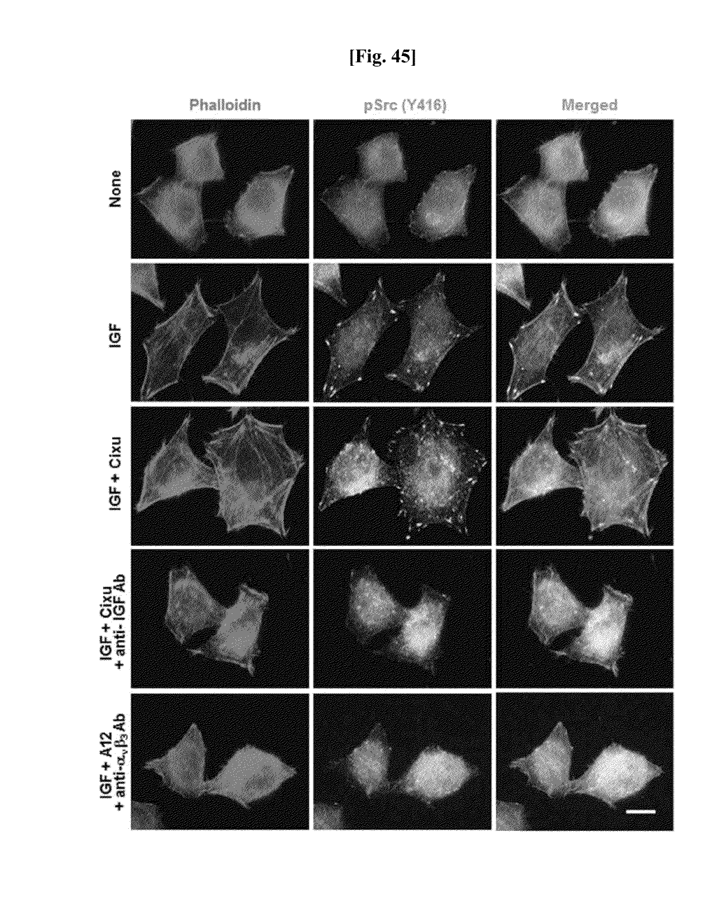

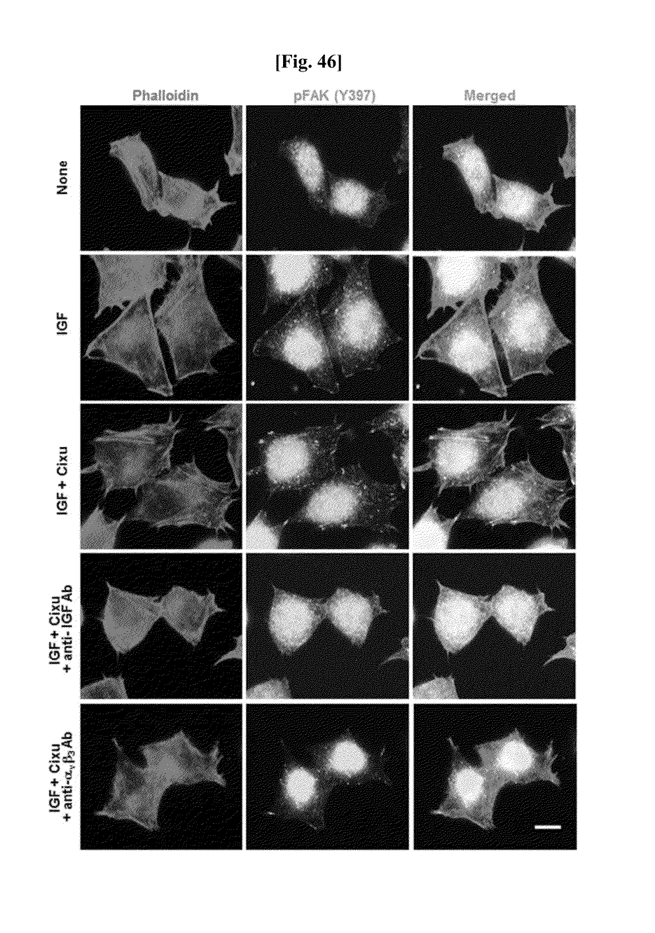

As shown in FIGS. 45 and 46, when IGF-1 was treated, Src and FAK phosphorylation was increased in a cell membrane, and cixutumumab did not suppress or somewhat increased such an action. In addition, the Src and FAK phosphorylation was lost when IGF-1 or an integrin β3 neutralizing antibody was simultaneously treated, and thus the results corresponding to the above descriptions could be confirmed.

Collectively, these data suggest that upon blockade of IGF binding to IGF-1R by cixutumumab treatment, IGF-1 binds to and activates integrin β3, leading to FAK/Src-mediated stimulation of EGFR and PI3K/Akt.

### Example 6

### Correlation of Degree of Integrin β3 Expression and Sensitivity of Cixutumumab

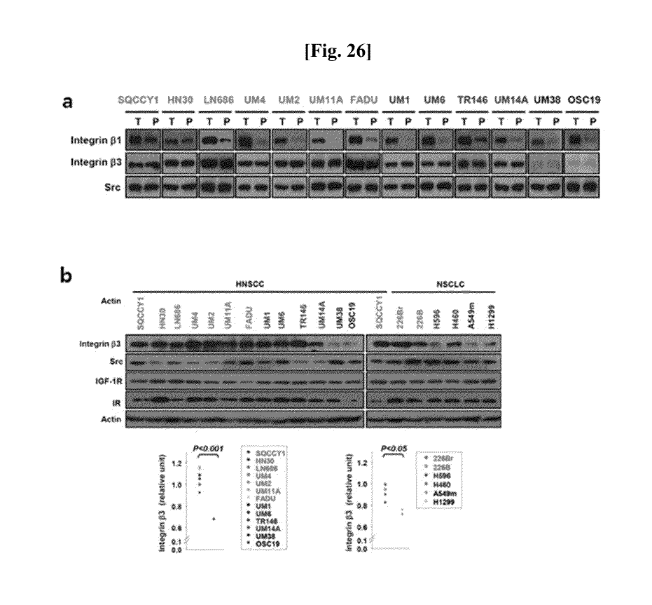

The present inventors analyzed analyzed the expression of integrin β3, Src, and IGF-1R by western blot analysis in HNSCC cell lines that had been grown in TCP and those grown in PCP. HNSCC and NSCLC cell lines grouped as cixutumumab-resistant group (in red), mildly sensitive group (in blue), and a sensitive group (in black) were cultured in TCP (T) or PCP (P). To analyze a protein expression level, western blot analysis was performed, and the results are quantitatively analyzed using densitometric analysis by digitizing the density of a blot. Integrin β3 expression level normalized by actin expression in each cell. And the results are shown in FIG. 26.

Consistent with a previous observation in cells under 3D culture conditions (Mizushima et al., 2009a), cixutumumab resistant (red), mildly sensitive (blue), and sensitive (black) HNSCC cell lines grown in 3D-mimic 2D culture conditions using PCP expressed decreased levels of integrin β1 protein. In contrast, expression level of integrin β3 was similar in 2D culture and 3D-mimic 2D culture conditions. In addition, the present inventors observed that, expression levels of Src and IGF-1R were not changed in the 3D-mimic 2D culture condition using PCPs (P), compared with those in the 2D culture condition (TCPs (T)). These findings suggest roles of integrin β3, src, and IGF-1R in cell groth in a 3D environment.

Afterwards, the present inventors confirmed a correlated expressions of integrin β3, Src, and IGF-1R with cixutumumab sensitivity. While the expression of the integrin β3 was significantly high in cixutumumab-resistant cell lines, No obvious correlation was observed between cixutumumab sensitivity and IGF-1R or Src expression level. Hence, expression of integrin β3 seemed to predict HNSCC and NSCLC cells' resistance to cixutumumab.

### Example 7

### Enhanced Effects of Cixutumumab on Inhibition of Cancer Cell Growth and Antitumor Activities when Used in Combination with Integrin β3 or Src Inhibitor

To test whether inactivation of integrin β3 or Src using the integrin β3-specific mAb (αβ3) or the small-molecule Src inhibitor (PP2) would prevent the activation of cixutumumab-mediated integrin/Src signaling, and thereby a cancer cell proliferation inhibitory effect of cixutumumab was increased.

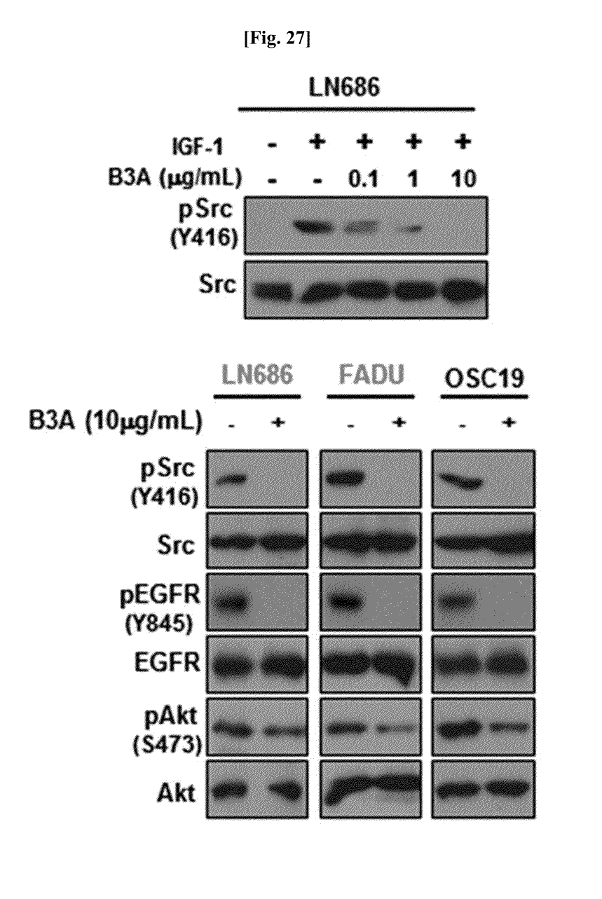

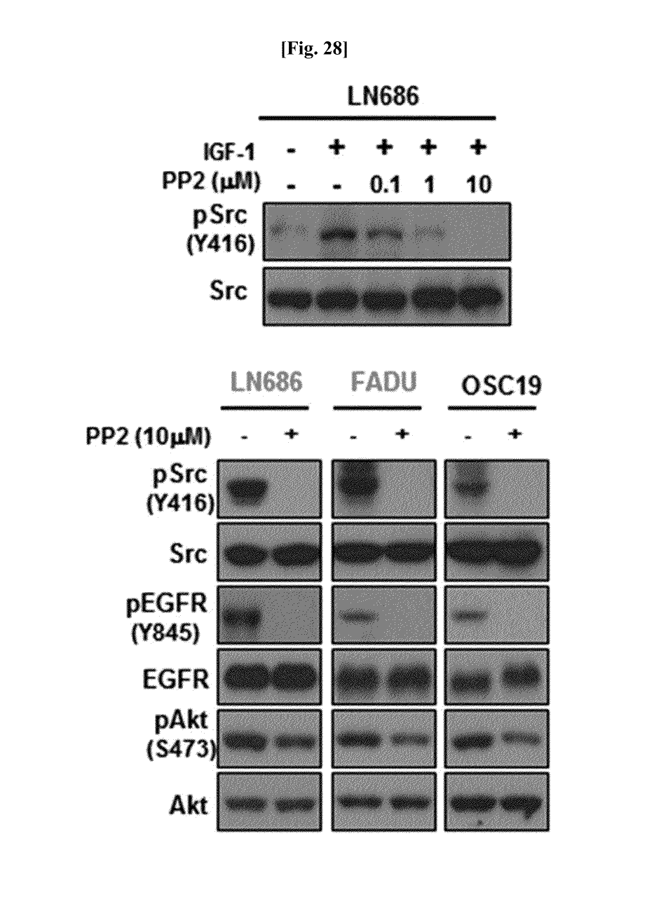

Specifically, cixutumumab-resistant cells (LN686 and FADU) and cixutumumab-sensitive cells (OSC19) grown in PCPs were treated with B3A (0.1-10 mg/ml) or PP2 (0.1-10 mM) for 1 hour prior to activation with IGF-1 (100 ng/ml). And the results obtained by analyzing protein expression through western blot analysis are shown in FIGS. 27 and 28.

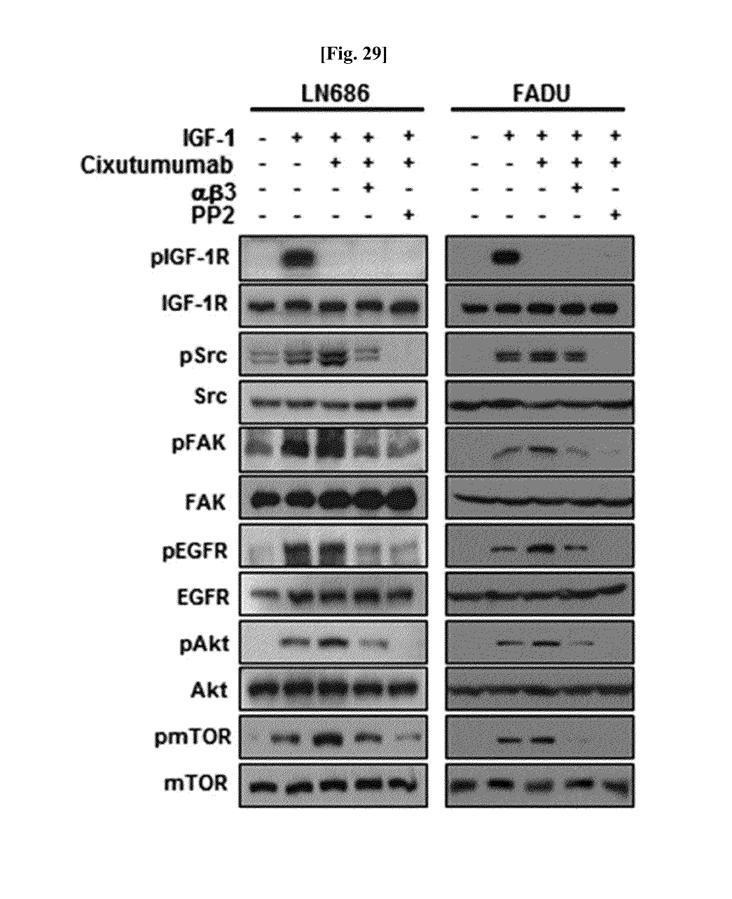

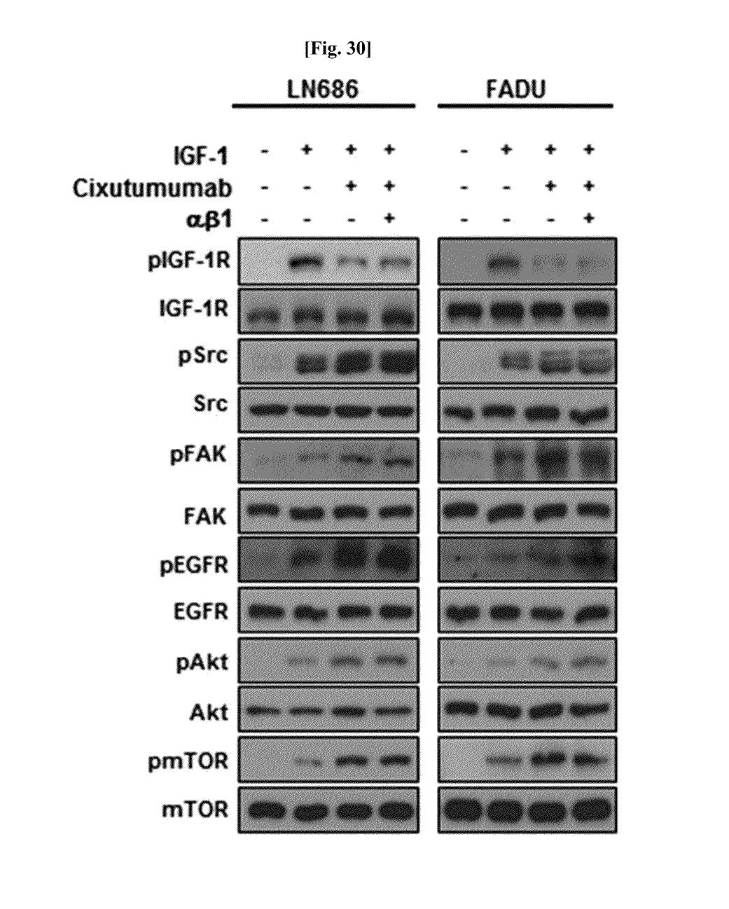

In addition, the LN686 and FADU cells grown in the PCPs were untreated or treated with cixutumumab (25 μg/ml) alone or in combination with anti-integrin β3 monoclonal antibody (7E3, 10 μg/ml), PP2 (10 μM), an anti-integrin β1 monoclonal antibody (A11B2, 10 μg/ml), Ad-EV or Ad-CSK (10 pfu/cell) for 7 days prior to stimulation with IGF-1 (100 ng/ml, 30 min), and to detect the proteins, western blot analysis was performed. And the results are shown in FIGS. 29 and 30.

As shown in FIGS. 27 and 28, both cixutumumab-resistant cells (LN686 and FADU) and cixutumumab-sensitive cells (OSC19) showed marked decreases in p-EGFR, p-Src, and p-Akt levels with no detectable changes in EGFR, Src, and Akt expression after 6 h treatment with integrin β3 mAb (B3A, 0.1-10 mg/ml) or PP2 (0.1-10 mM). In addition, as shown in FIG. 29, Treatment with integrin β3 mAb (100 μg/ml) or PP2 (10 μM) almost completely blocked cixutumumab-induced increases in p-Src, p-FAK, p-EGFR, p-Akt, and p-mTOR LN686 or FaDu cells.

In contrast, inactivation of β1 integrin had no influence on the Src, FAK, EGFR, Aid, and mTOR phosphorylation induced by cixutumumab.

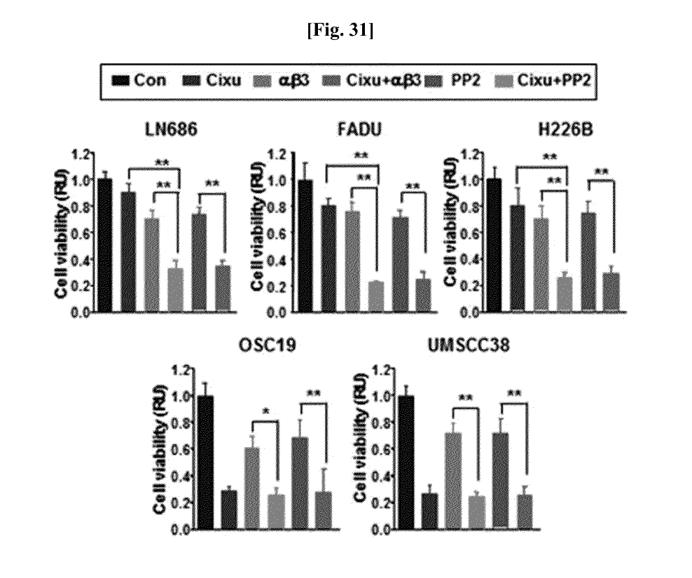

In addition, the LN686, FADU, H226B, OSC19, and UMSCC38 cells grown in PCPs were untreated or treated with cixutumumab (25 μg/ml), anti-integrin β3 mAb (αβ3, 10 μg/ml), PP2 (10 μM), or a combination thereof for 7 days. Cell proliferation was analyzed by MTS assay, and the results are shown in FIG. 31. Each bar represents the mean±SD of 6 wells treated with the same sample in one representative experiment.

As shown in FIG. 31, combined treatment with cixutumumab and integrin β3 mAb or PP2 showed significantly augmented antiproliferative activities of cixutumumab in the cixutumumab-resistant cells (LN686, FADU, and H226B), but not in the cixutumumab-sensitive cells (OSC19 and UMSCC38).

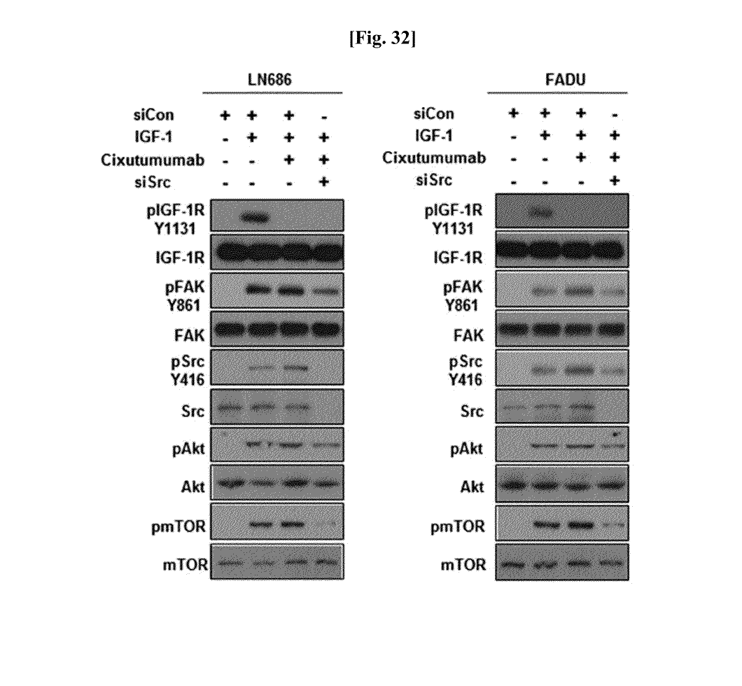

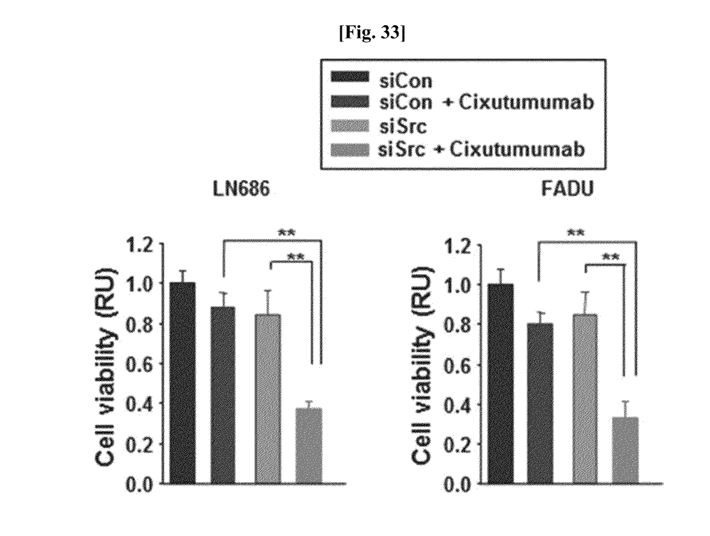

In addition, specific blockade of Src through transfection with Src siRNA almost completely blocked the IGF-dependent effects of cixutumumab on phosphorylation of Src, FAK, Aid, and mTOR (FIG. 32) and significantly augmented suppression of cancer cell growth induced by cixutumumab (FIG. 33) in LN686 and FADU cells grown in PCPs.

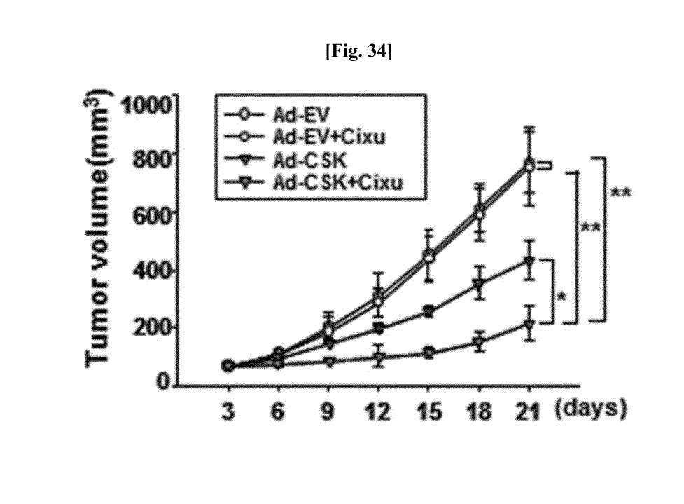

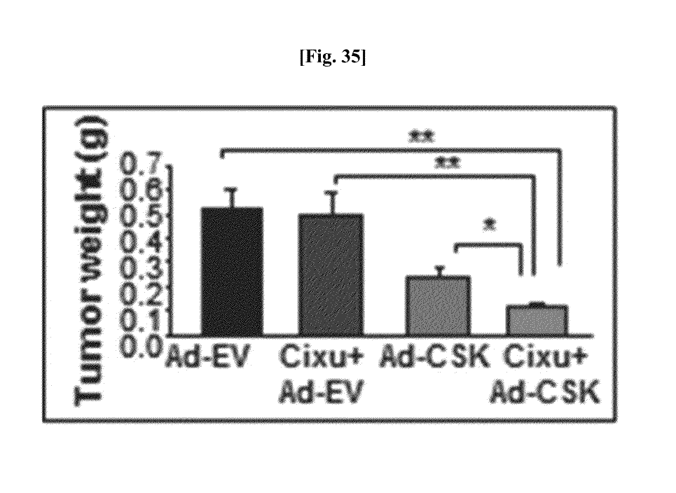

To further determine the effects of inhibiting integrin β3/Src signaling on antitumor activities of cixutumumab, an adenovirus expressing c-Src tyrosine kinase that phosphorylates the autoinhibitory tyrosine 527 was used (Yeatman, 2004). Specifically, after a xenograft model (n=8) was made by implanting LN686 cells into a Athymic nude mouse, cixutumumab (25 mg/kg, abdominal injection), Ad-EV (3×10¹¹ pfu, intratumoral injection), Ad-CSK (3×10¹¹ pfu, intratumoral injection), or a combination thereof was injected twice a week for indicated time, and the results are shown in FIGS. 34 and 35 (The bar represents the mean±SD; *P<0.05, **P<0.01.)

Ad-CSK reduced cixutumumab-induced Src phosphorylation in LN686 cells. Combined treatment with cixutumumab and Ad-CSK LN686-xenografted tumors significantly more effectively than did treatment with cixutumumab or Ad-CSK alone (FIG. 34). After 21 days, compared to a control group, the mean tumor weight in the individual or combined treating group was respectively 21% (P<0.001), 96%, or 46% (P<0.01), respectively (FIG. 35). These results suggested that that inactivation of integrin β3/Src signaling overcome resistance to cixutumumab in IGF-1R and integrin β3 dual-positive HNSCC cells.

Unlike single cell lines used in current studies, since actual cancer cells are composed of cells having various characteristics, to substantially reflect this, an influence on antitumor effects of cixutumumab was confirmed when human head and neck cancer tissues were obtained and implanted into nude mice, and cixutumumab and an integrin β3 inhibitor or Src inhibitor were administered alone or in combination. Specifically, to block integrin β3, the present inventors used siRNAs or integrin β3-neutralized mAb (αβ3).

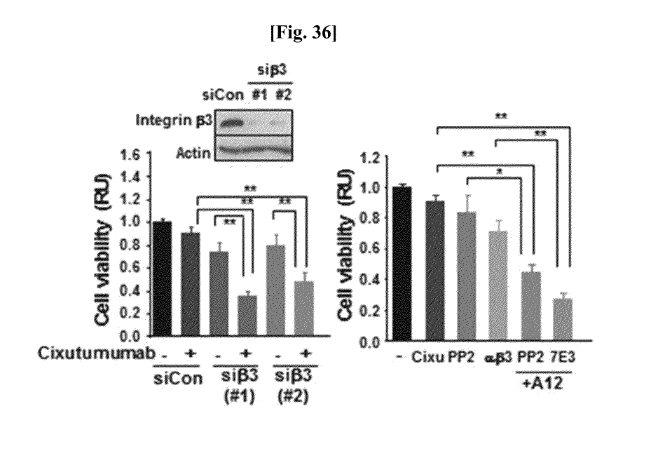

Primary cultured cells isolated from tumor tissues of HNSCC patients were transfected with either of two specific siRNAs (#1 and #2) against integrin β3 (siβ3) or control siRNA (siCon) (80 nM) for 24 h (FIG. 36, left). Suppression of integrin β3 by the siRNA transfection was confirmed by Western blot analysis (FIG. 36, top). Cells were treated with cixutumumab (25 μg/ml), PP2 (10 μM), and integrin β3 mAb (αβ3, 10 μg/ml), or a combination thereof for 72 h (FIG. 36, right). The inhibitory action on cancer cell growth was measured by MTS assay. Data was represented as an mean±SD of experiments (n=7, **P<0.05).

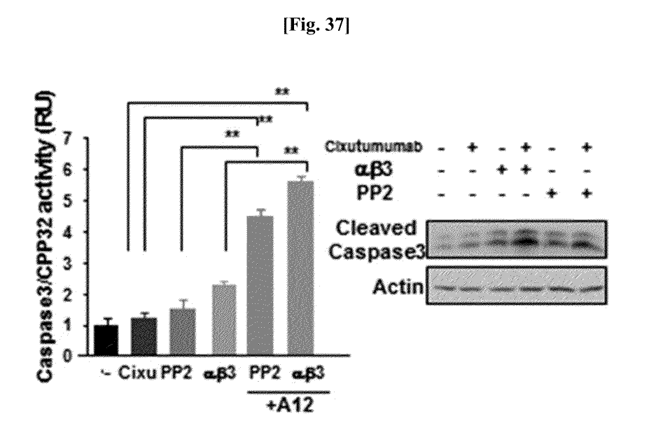

The cell lysates used for the caspase-3 was colormetric assay (FIG. 37, left), and western blot analysis (FIG. 37, right). By comparing whether or not an absorbance was increased due to p-nitroaniline produced by caspase activation (measured at 405 nm) (left), or comparing levels of the cleaved caspase-3 protein.

Consequently, as shown in FIG. 36, two different siRNAs that almost completely suppressed integrin β3 expression significantly augmented the cancer cell growth inhibitory effect of cixutumumab on the HNSCC cells grown in PCPs. And as shown in FIG. 37, Similar stimulation to the cancer cell growth inhibitory effect of cixutumumab were observed when Src was suppressed using mAb of integrin β3 or an inhibitor (PP2).

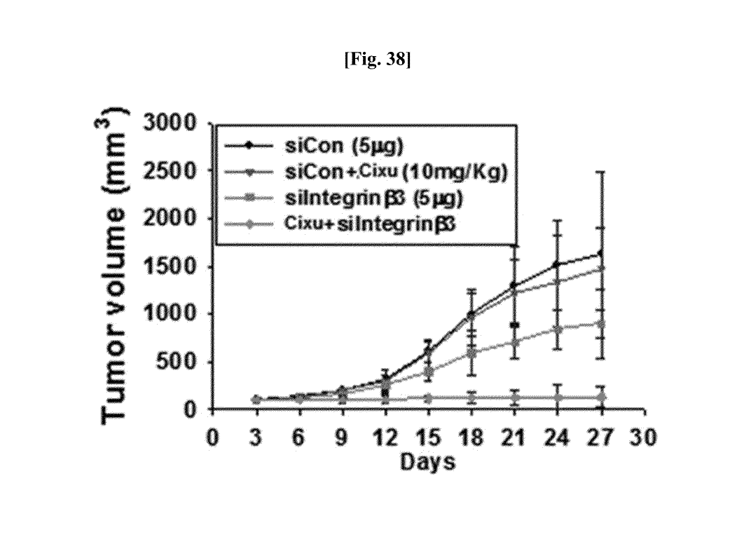

To reconfirm such results in vivo, Athymic nude mice were transplanted with tumor tissue (2 mm³) from HNSCC patients, and cixutumumab (10 mg/kg, ip, 1/wk×3) was administered in combination with control siRNA (5 μg, iv, 2/wk×3) or integrin β3 siRNA (5 μg, iv, 2/wk×3), or in combination with dasatinib (10 mg/kg, po, daily). siRNA was administered to the mouse using a liposome as previous study (Verma et, al., 2008). Test data are represented as mean tumor volume±SD for indicated times, and shown in FIGS. 38 to 41.

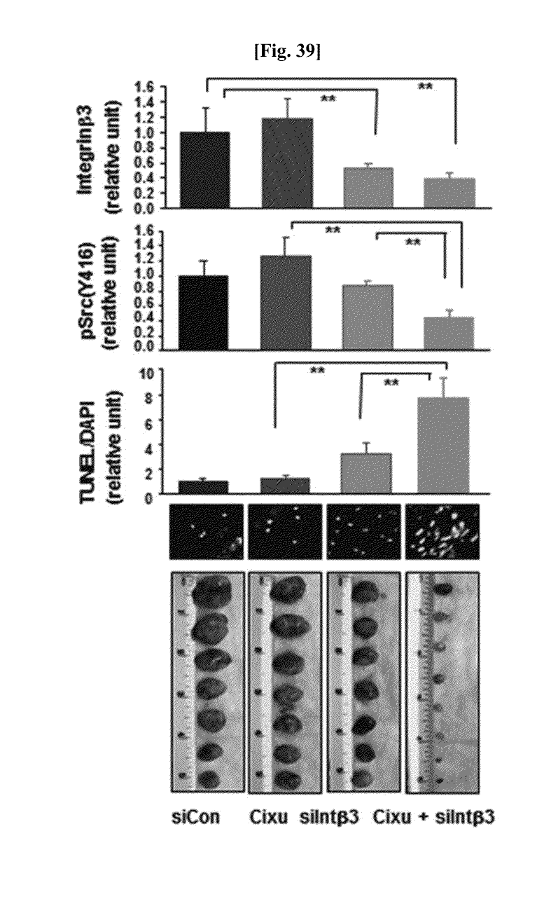

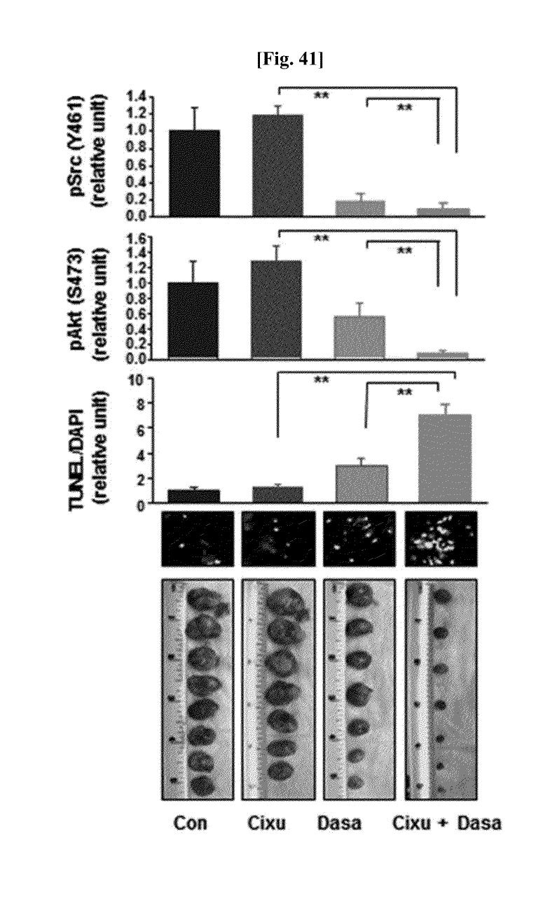

In addition, a protein was isolated from tumor and analyzed for integrin β3, pSrc, and pAkt expressions. Moreover, TUNEL staining was performed on a tissue fragment, thereby confirming an increase in apoptosis, and the results are shown in FIGS. 39 and 41 (*P<0.05; **P<0.01; ***P<0.001).

As shown in FIG. 38, when cixutumumab was administered in combination with integrin β3 siRNA, the suppression of tumor growth was quite significantly decreased, and such an action occurred due to the suppression of Src phosphorylation and an increase in cell death through apoptosis (FIG. 39). In addition, as the integrin β3 expression was significantly decreased in the integrin β3 siRNA treated group, it was confirmed that siRNA worked effectively in the tumor tissues.

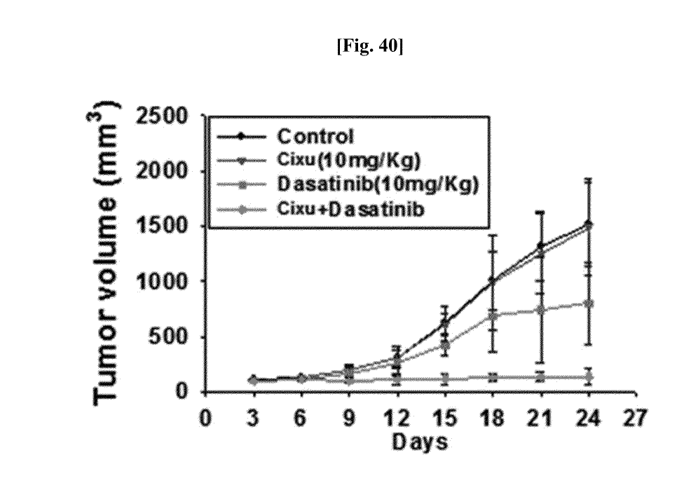

Referring to FIG. 40, tumor growth was quite significantly decreased among the control group, the cixutumumab-only treated group, the dasatinib-only treated group, and the cixutumumab and dasatinib-simultaneously treated group. In addition, as shown in FIG. 41, in the tumor obtained from the cixutumumab and dasatinib-simultaneously treated group, Akt and Src phosphorylation was significantly decreased, and an indicator of the increase in apoptosis, that is, TUNEL staining, was also apparently increased, and the tumor growth was apparently decreased.

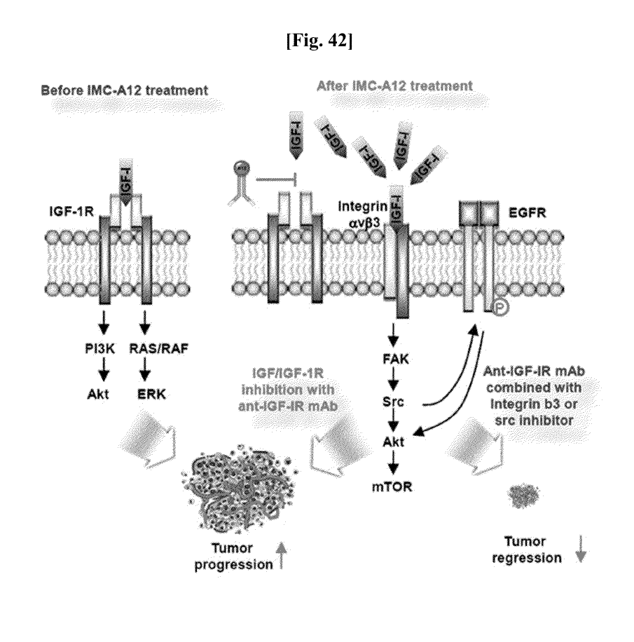

The above results are summarized as shown in FIG. 42. It is assumed that when the binding of IGF to IGFR was suppressed by cixutumumab treatment, IGF not binding to IGFR bound to integrin β3, to induce integrin-mediated cell growth stimulation and activation of death suppressing signal transduction, resulting in resistance to antitumor effects of cixutumumab.

While the invention has been shown and described with reference to certain exemplary embodiments thereof, it will be understood by those skilled in the art that various modifications in form and details may be made therein without departing from the spirit and scope of the invention as defined by the appended claims.

1. A pharmaceutical composition to overcome resistance to a targeted anticancer drug, comprising:
at least one selected from the group consisting of an integrin β3 neutralizing antibody, integrin β3 siRNA, Src inhibitor, and Src siRNA as an active ingredient.

2. The composition according to claim 1, wherein the targeted anticancer drug is an insulin-like growth factor-1 receptor (IGF-1R) targeted anticancer drug.

3. The composition according to claim 1, wherein the targeted anticancer drug is an anti-IGF-1R monoclonal antibody.

4. The composition according to claim 1, wherein the integrin β3 neutralizing antibody, integrin β3 siRNA, Src inhibitor, or Src siRNA inhibits αIGF-dependent effects of the anti-IGF-1R monoclonal antibody.

5. The composition according to claim 1, wherein the integrin β3 neutralizing antibody, integrin β3 siRNA, Src inhibitor, or Src siRNA inhibits phosphorylation of Src, EGFR, Akt, FAK, or mTOR.

6. The composition according to claim 1, wherein the integrin β3 neutralizing antibody, integrin β3 siRNA, Src inhibitor, or Src siRNA induces dephosphorylation of p-Src, p-EGFR, p-Akt, p-FAK, or p-TOR.

7. The composition according to claim 5, wherein the Src phosphorylation occurs at a position of tyrosine 416 (tyrosine 416, Y416).

8. The composition according to claim 5, wherein the EGFR phosphorylation occurs at a position of tyrosine 1068 (tyrosine 1068, Y1068) or tyrosine 845 (tyrosine 845, Y845).

9. The composition according to claim 5, wherein the FAK phosphorylation occurs at a position of tyrosine 861 (tyrosine 861, Y861).

10. An anticancer supplement, comprising:
at least one selected from the group consisting of an integrin β3 neutralizing antibody, integrin β3 siRNA, Src inhibitor, and Src siRNA as an active ingredient, and which enhance antiproliferative activities of an anticancer drug.

11. The anticancer supplement according to claim 10, wherein the anticancer drug is an IGF-1R targeted anticancer drug.

12. The anticancer supplement according to claim 10, wherein the anticancer drug is an anti-IGF-1R monoclonal antibody.

13. The anticancer supplement according to claim 10, wherein the integrin β3 neutralizing antibody, integrin β3 siRNA, Src inhibitor, or Src siRNA inhibits the IGF-dependent effects of an anti-IGF-1R monoclonal antibody.

14. A method of enhancing antitumor effects, comprising:
administering at least one selected from the group consisting of an integrin β3 neutralizing antibody, integrin β3 siRNA, Src inhibitor and Src siRNA, or a pharmaceutically acceptable salt thereof to an individual in combination with a targeted anticancer drug.

15. The method according to claim 14, wherein the targeted anticancer drug is an IGF-1R targeted anticancer drug.

16. The method according to claim 14, wherein the targeted anticancer drug is an anti-IGF-1R monoclonal antibody.

17. A method of predicting resistance to anticancer drugs, comprising:
measuring expression of integrin β3.
